(19) United States
(12) Patent Application Publication
(10) Pub. No.: US 2022/0392590 A1
(43) Pub. Date: Dec. 8, 2022

(54) GRANULAR DATA UPDATE SHARING

(71) Applicant: Apple Inc., Cupertino, CA (US)

(72) Inventors: Lucas O. Winstrom, Palo Alto, CA (US); Blanca Isabel C. Villanueva, Berkeley, CA (US); Glen R. DePalma, Fremont, CA (US); Reza Azimi, San Francisco, CA (US)

(73) Assignee: Apple Inc., Cupertino, CA (US)

(21) Appl. No.: 17/831,989

(22) Filed: Jun. 3, 2022

Related U.S. Application Data
(60) Provisional application No. 63/197,957, filed on Jun. 7, 2021.
(60) Provisional application No. 63/330,572, filed on Apr. 13, 2022.

(51) Int. Cl. G16H 10/60; G16H 50/70

(52) U.S. Cl. CPC G16H 10/60; G16H 50/70

(57) ABSTRACT

Techniques for identifying change points in health data are described herein. Health data during a first time sub-window is compared to health data from a second time sub-window. The health data is evaluated with respect to a set of change point criteria to determine that a first change is a first change point in the health data. A notification including information about the change point and information about a second change point is generated.

### CROSS REFERENCE TO RELATED APPLICATIONS

This application is claims priority under 35 U.S.C. § 119(e) to U.S. Provisional Application No. 63/330,572, filed on Apr. 13, 2022, and claims priority to U.S. Provisional Application No. 63/197,957, filed Jun. 7, 2021. The contents of these applications are hereby incorporated by reference in their entirety for all purposes.

### BACKGROUND

Electronic devices, especially portable electronic user devices, are quickly becoming ubiquitous in every modern society. Such devices can be used to collect and store personal information, such as health data, about a user.

### BRIEF SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a computer-implemented method. The computer-implemented method includes responsive to a first user input at a first user device, identifying a second user associated with a second user device to receive health data updates associated with a first user of the first user device. The computer-implemented method also includes responsive to a second user input at the first user device, identifying a set of authorization identifiers that each represent a type of health data update to be shared with the second user device. The computer-implemented method also includes generating a sharing request that identifies the set of authorization identifiers and a unique identifier that identifies the second user. The computer-implemented method also includes sharing information associated with the sharing request with the second user device. Other embodiments of this aspect include corresponding computer systems, apparatus, and computer programs recorded on one or more computer storage devices, each configured to perform the actions of the methods.

Another general aspect includes another computer-implemented method. The computer-implemented method includes receiving, at a first user device associated with a first user, a sharing request associated with sharing health data of a second user associated with a second user device with the first user device. The computer-implemented method also includes requesting, from a server, transaction data stored by a server. The computer-implemented method also includes in response to requesting the transaction data, receiving, from the server, a first transaction package including first one or more health data updates associated with the health data of the second user. The computer-implemented method also includes extracting the first one or more health data updates from the first transaction package. The computer-implemented method also includes populating a user interface of the first user device with information associated with the first one or more health data updates. Other embodiments of this aspect include corresponding computer systems, apparatus, and computer programs recorded on one or more computer storage devices, each configured to perform the actions of the methods.

Another general aspect includes another computer-implemented method. The computer-implemented method includes determining a first change point in a first type of health data. The computer-implemented method also includes determining a second change point in a second type of health data that is correlated with the first type of health data. The computer-implemented method also includes in accordance with a determination that the first change point meets a first set of notification criteria, generating a notification that includes first information associated with the first change point. The computer-implemented method also includes in accordance with a determination that the second change point meets a second set of notification criteria, including second information associated with the second change point in the notification. Other embodiments of this aspect include corresponding computer systems, apparatus, and computer programs recorded on one or more computer storage devices, each configured to perform the actions of the methods.

Another general aspect includes another computer-implemented method. The computer-implemented method includes accessing first health data of a first data type collected during a time window. The computer-implemented method also includes comparing a first quantity of the first health data associated with a first time sub-window of the time window with a second quantity of the first health data associated with a second time sub-window of the time window to identify a first change in the first health data. The computer-implemented method also includes in accordance with a first evaluation of the first and second quantities of the first health data with respect to a first set of change point criteria, determining that the first change is a first change point in the first health data. The computer-implemented method also includes generating a notification that includes information about the first change point. The computer-implemented method also includes including information, in the notification, about a second change point in second health data when a second evaluation of the second health data with respect to a second set of change point criteria is indicative of the second change point. Other embodiments of this aspect include corresponding computer systems, apparatus, and computer programs recorded on one or more computer storage devices, each configured to perform the actions of the methods.

### BRIEF DESCRIPTION OF THE DRAWINGS

FIG. 1 illustrates a block diagram and a flowchart showing an example process for conducting sharing of health data updates among user devices, according to at least one example.

FIG. 2 illustrates a flowchart showing a process for conducting sharing of health data updates among user devices, according to at least one example.

FIGS. 3A-3E illustrate example user interfaces presented on a user device for conducting sharing of health data updates among user devices, according to various examples.

FIG. 4 illustrates a diagram showing graphs for identifying changes in health data and providing opportunistic notifications with user devices, according to at least one example.

FIG. 5 illustrates a flowchart of a process for conducting sharing of health data updates among user devices, according to at least one example.

FIG. 6 illustrates a flowchart of a process for conducting sharing of health data updates among user devices, according to at least one example.

FIG. 7 illustrates a flowchart of a process for identifying changes in health data and providing opportunistic notifications with user devices, according to at least one example.

FIG. 8 illustrates a flowchart of a process for identifying changes in health data and providing opportunistic notifications with user devices, according to at least one example.

FIG. 9 illustrates a flowchart of a process for identifying changes in health data and providing opportunistic notifications with user devices, according to at least one example.

FIG. 10 illustrates an example architecture or environment configured to implement techniques relating to conducting sharing of health data updates among user devices and identifying changes in health data, according to at least one example.

FIG. 11 illustrates an electronic device for implementing techniques relating to conducting sharing of health data updates among user devices and identifying changes in health data, according to at least one example.

FIG. 12 illustrates a simplified block diagram including components of an example electronic device for implementing techniques relating to conducting sharing of health data updates among user devices and identifying changes in health data, according to at least one example.

FIG. 13 illustrates a simplified diagram including example electronic devices for implementing techniques relating to conducting sharing of health data updates among user devices and identifying changes in health data, according to at least one example.

FIG. 14 illustrates an example electronic device for implementing techniques relating to conducting sharing of health data updates among user devices and identifying changes in health data, according to at least one example.

### DETAILED DESCRIPTION

In the following description, various examples will be described. For purposes of explanation, specific configurations and details are set forth in order to provide a thorough understanding of the examples. However, it will also be apparent to one skilled in the art that the examples may be practiced without the specific details. Furthermore, well-known features may be omitted or simplified in order not to obscure the example being described.

Examples of the present disclosure are directed to, among other things, methods, systems, devices, and computer-readable media that provide mechanisms for establishing and maintaining connections for automated sharing of updates to health data between user devices. Unlike conventional sharing arrangements, the techniques described herein enable granular user control over what data types are shared and with whom. For example, a user may select from a set of predefined health data types, which may include types relating to data generated using sensors of the user device or those of one or more related devices, types relating to data derived from instances of the user's health record, and/or types relating to data input by the user. When a sharing request is sent to a recipient device, a user of the recipient device may see what specific data types will be shared if the request is accepted.

Once the sharing request has been accepted, the initiating user device may collect the specific data types for a given period, bundle them together into a single transaction for the given period, and share them with a cloud-based storage service. The cloud-based storage service, which may be implemented by a service provider, may provide for secure storage of the transactions in a storage zone that is accessible to the initiating user device and the recipient user device. When updates are sent from multiple user devices associated with the sending user (e.g., a smartphone and a tablet), the recipient user device may use predefined logic to pick which transaction is most suitable. This may include, in some examples, comparing the recency of the transactions, whether the transactions include health data derived from an accessory device such as a smartwatch, a version of an app on the user device that collected the health data, and the like. The recipient user device may then download the transaction and retrieve the health data updates. Use of a transaction-based system for health update sharing, as described herein, may ensure that the initiating user and the recipient user are viewing a complete and consistent view of the health data that are being shared. Conventional sharing techniques, which did not utilize such a transaction-based system such as the one described herein, could result in some data being stale or inconsistent, and the user(s) would be unaware. This can lead to poor user experiences, not to mention the risk of users taking health-related actions using inconsistent or stale health data.

Depending on changes in the data compared to other health data updates, the recipient user device may trigger notifications relating to the health data updates. These notifications may indicate that new health data updates are available, identify changes in the health data, identify whether the health data is indicative of a trend in one direction or the other, and indicate any other aspect of the health data update.

In some examples, the initiating user device may process the health data (e.g., raw or post-processed) on-device to determine whether data of a particular data type is representative of a trend, while also including information about the trend in the health data updates. In some examples, the recipient device may process the health data to determine whether a trend is present. The rules for determining that a trend is present may be specific for a particular data type. In some examples, correlations between data types may be maintained such that when a notification about a first trend in a first data type is triggered, the system may determine whether a second trend in a second related data type is likely to trigger in the near future. This determination may enable the generation and delivery of a single notification about the trends, rather than a first notification about the first trend now and a second notification about the second trend in the near future. In some examples, this may be referred to opportunistic notifications, which may reduce network traffic, while also improving the user experience on-device by reducing the likelihood of notification fatigue. In some examples, the notifications about the trends may also be managed by invoking a back-off period that determines whether change points in the data are too close together and/or in the same direction. In this example, the system may refrain from generating a new notification.

Turning now to a first particular example, in this example an initiating user device may send an offer to share health data updates with a recipient user device associated with a recipient user. The health updates may be associated with the initiating user. The process of generating the offer may be managed by an application executing on the initiating user device. For example, a user selection user interface may be presented at the initiating user device. This user interface may be used by the initiating user to select the recipient user to receive the health data updates from a set of users. Once selected, an authorization identifier user interface may be presented at the initiating user device. This user interface may be used by the initiating user to authorize which types of data will be shared with the recipient user device. Once identified, the initiating user device may store authorization identifiers that identify the individual data types in association with an identifier of the recipient user. The authorization identifiers may be used to capture the correct data for sharing and to generate a sharing request to be sent to the recipient user device using the identifier. If the sharing request is accepted by the recipient user—using the recipient user device—the initiating user device may receive confirmation, which may trigger a routine to begin collecting health data updates. In some examples, the initiating user device may bundle health data updates for different data types into a single transaction for a given time period (e.g., hour, few hours, day, week, month, multiple months, a year, multiple years, etc.). This transaction then may be sent to a cloud-based storage service and stored thereby in a storage zone to which the recipient user device has access. The recipient user device may obtain the transaction from the cloud-based storage service in any suitable manner and at any suitable cadence.

Turning now to a second particular example, in this example a recipient user device may receive a sharing request from an initiating user device. The sharing request may constitute an offer by an initiating user associated with the initiating user device to share health data updates of particular types with a recipient user associated with the recipient user device. Once the sharing request has been accepted, e.g., by the recipient user indicating as such via an acceptance user interface, the recipient user device may request transaction data associated with the initiating user from a cloud-based storage service. Responsive to this request, the recipient user device may receive the requested transaction data, which may include health data updates associated with the initiating user. In some examples, the recipient user device may download multiple transaction packages, each being uniquely identified. For example, the initiating user may operate more than one user device associated with the same user profile. In this example, a first initiating user device (e.g., a smartphone) may generate a first transaction package including first health data updates and a second initiating user device (e.g., a tablet) may generate a second transaction package including second health data updates. The recipient user device may download both transaction packages and, using a set of prioritization rules, select one transaction package for use. Once the recipient user device has selected which transaction package to use, the recipient user device may extract the health data updates from the transaction package. Following this, the recipient user device may generate notifications relating to the updates, populate a user interface of the recipient user device with information about the updates, and perform any other suitable operations with respect to the updates.

Turning now to a third particular example, in this example a user device may be used to identify changes in the health data maintained or otherwise tracked by the user device, and to determine whether to inform a user associated with the user device (and the health data) about the changes. The changes of interest here relate to changes in patterns of data over time. For example, a user used to take X number of steps per day and for the last ten days has been taking significantly fewer than X number of steps. A change in the data may be referred to as a change point. When the change point constitutes a change to the pattern, it may be referred to as a trend. The user device may determine whether to inform the user about a change point and/or a trend, depending on a number of factors. For example, if the change point occurs too close to an earlier change point in the same data type, the user device may refrain from generating a notification. If the change point is in the same direction as a previous change point that was notified on, the user device may also refrain from sending another notification. In some examples, the user device may also look for opportunities to send notifications about correlated data that do not yet, but are likely to, constitute a trend, within some given time period. In this example, the user device may decide to bundle the updates into a single notification.

Turning now to a fourth particular example, in this example a user device may be used to identify changes in health data over time (e.g., during a defined time window), and to determine whether to inform a user associated with the user device (and the health data) about the changes. Like the previous example, the changes in health data may be the result of changes in behaviors of the user. To begin, the user device may collect or otherwise access health data. For example, the user device may include onboard sensors that collect at least a portion of the health data, the user device may obtain another portion of the health data from an accessory device that includes sensors to collect health data. In some examples, the user device may obtain health data from a device associated with a different user, e.g., using the health update sharing techniques described herein. In any event, once the user device has access to health data, which may be of various data types (e.g., steps, flights of stairs, workout minutes, minutes standing, heartrate, etc.), the user device may evaluate the health data using various dynamically sized time sub-windows that are shorter in length than the time window. Evaluating the health data may include evaluating the health data in accordance with a first set of change point criteria. The first set of change point criteria may be used to identify a change point in the data, e.g., a division of the time window into two sub-windows that each represent sufficiently different data values. For example, for a particular data type such as steps taken, the first sub-window may be a pre-change point sub-window that identifies that the user has generally taken on average around 5000 steps per day, and the second sub-window may be a post-change point sub-window that identifies that the user has generally taken on average around 7000 steps per day. The change point may be defined as the time between the two sub-windows. In some examples, the first set of change point criteria may also include a minimum threshold, which may correspond to medical relevance. For example, the minimum threshold, which may be specific to data type, time period, and/or whether the criteria are being evaluated as a first change point or a second change point, may represent a minimum difference between the data in the two sub-windows. Thus, in addition to the change point criteria including statistical measures, the change point criteria may also include minimum thresholds. These thresholds may be determined by medical professionals (e.g., using empirical methods) to be indicative of medically relevant change. Thus, the minimum threshold may assure that the change is medically relevant to the user and the statistical measures may assure that the change is statistically relevant. When a change point is registered, the user device may generate a notification about the change point and may then determine whether there are other changes in the health data that should be included in the notification. This can include determining whether there are other changes points in other data types based on the first set of change point criteria. The user device may also look for change points for other data types based on a second set of change point criteria. The second set of change point criteria may be more relaxed than the first set of change point criteria. The second set of change point criteria may also include one or more minimum thresholds, which may be specific to the data type, time period, and/or whether the change point is a first change point or a second change point relating to the first change point. In this manner, the second set of change point criteria may be useful for identifying data that is trending towards a change point under the first set of change point criteria, but would not otherwise meet the first set of change point criteria. Evaluating the other data types in this manner may enable opportunistically notifying the user about health updates of different data types that are correlated, even when one or more data types are less indicative of the trend (e.g., not as far along as a first data type). In some examples, fulfillment of the first set of change point criteria based on a first data type may mean the user should definitely be notified about the first change point, while fulfillment of the second set of change point criteria with respect to a second data type may mean the user should be notified about the second change point so long as the user device is already notifying the user about the first change point. Thus, once the user device has identified a data type that meets the first set of change point criteria, the user device will look for other data types that meet the second set of change point criteria that can be opportunistically included with a notification about the first change point.

The examples described herein address a number of technical problems and provide for a number of technical improvements. In some examples, these improvements additionally improve the functioning of various components of a system in which the techniques are implemented. The techniques described herein provide for sharing of health data updates in a manner that minimizes network traffic, as compared to conventional systems. For example, rather than sharing all health data generated or otherwise obtained from an initiating user device as was conventionally done, the techniques described herein provide for methods of user selection of what data will be shared at a data type level. This results in much smaller data transfer demands, as compared to conventional approaches. Additionally, because the techniques described herein allow for customized sharing, resources devoted to storing the health data updates at the initiating user device, a cloud-based storage service, and a recipient user device are all reduced. Moreover, because a user may select what health data is shared, the user maintains control over their privacy as it relates to sensitive data. When sharing does occur, the data is shared between the user devices (and the cloud-based storage service) in a manner that keeps the cloud-based storage service from being able to read the health data, much less associate the health data with the particular user to which they belong.

Additionally, because the techniques described herein reduce the number of notifications provided to a user for trending correlated data types, user devices realize additional technical improvements including increased battery life, reduced bandwidth usage, and memory conservation. In some examples, displaying fewer notifications may reduce the effects of alarm fatigue, resulting in more meaningful delivery of notifications. Moreover, the notifications that are displayed may include selectable user interface objects that have visual characteristics based on health-related data, which may provide the user with feedback regarding the data types included in the health-related data. Providing improved visual feedback enhances the operability of the device and makes the user device interface more efficient (e.g., by helping the user to provide proper inputs and reducing user mistakes when operating/interacting with the device) which, additionally, reduces power usage and improves battery life of the device by enabling the user to use the device more quickly and efficiently.

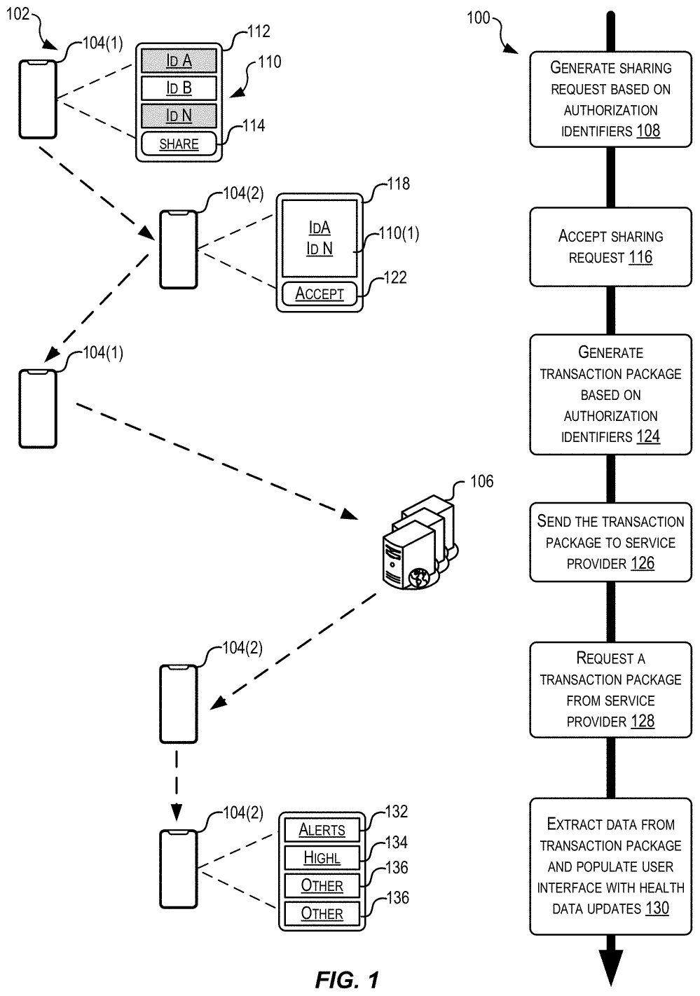

Turning now to the figures, FIG. 1 illustrates a block diagram 102 and a flowchart showing a process 100 for conducting sharing of health data updates among user devices, according to at least one example. The diagram 102 includes one or more user devices 104 (referred to herein as user devices 104 and user device 104). In particular, the diagram 102, which is indicative of a computer architecture that may be used to implement the techniques described herein, includes an initiating user device 104(1) and a recipient user device 104(2). Both user devices 104(1) and 104(2) are illustrated as handheld portable user devices such as smartphones. As described herein, an example user device 104 may be any suitable user device such as a smartphone, tablet, media player, laptop, wearable device, and the like. In some examples, the user device 104 may include one or more applications, which may include custom-built algorithms and other logic, to enable performance of at least some of the techniques described herein. The user device 104 may also include storage media for storing computer-executable instructions (e.g., that make up the application) and other data such as described herein. The user devices 104 may be operated by users.

The diagram 102 also includes a service provider 106, which is any suitable combination of computing devices such as one or more server computers. The service provider 106 may include virtual resources, capable of performing the functions described with respect to the service provider 106. For example, the service provider 106 may include one or more different servers and/or services dedicated to handling different aspects of processing user requests relating to sharing health data updates, identifying trends, and the like. The service provider 106 may maintain profiles for the users. For example, the service provider 106 may maintain a user profile relating to digital services offered by the service provider (e.g., messaging, cloud backup, music streaming, health, fitness, contacts, data services, etc.).

FIGS. 1, 2, and 5-9 illustrate example flow diagrams showing processes 100, 200, 500, 600, 700, 800, and 900, according to at least a few examples. These processes, and any other processes described herein, are illustrated as logical flow diagrams, each operation of which represents a sequence of operations that can be implemented in hardware, computer instructions, or a combination thereof. In the context of computer instructions, the operations may represent computer-executable instructions stored on one or more non-transitory computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, some, any, or all of the processes described herein may be performed under the control of one or more computer systems configured with specific executable instructions and may be implemented as code (e.g., executable instructions, one or more computer programs, or one or more applications) executing collectively on one or more processors, by hardware, or combinations thereof. As noted above, the code may be stored on a non-transitory computer-readable storage medium, for example, in the form of a computer program including a plurality of instructions executable by one or more processors.

The process 100 begins at block 108 by the initiating user device 104(1) generating a sharing request based on authorization identifiers 110. As part of generating the sharing request, the initiating user device 104(1) may present a user selection interface 112. The user selection interface 112 may present the authorization identifiers 110, which may identify corresponding health data types. For example, the authorization identifiers 110 may include ID A, ID B, and ID N, which may correspond to any suitable number of authorization identifiers 110 that identify any suitable number of health data types. In the illustrated example, the user has selected the authorization identifiers 110 corresponding to ID A and ID N. This may mean that the user intends to share data types represented by ID A and ID N. The user selection interface 112 may identify additional information about the data types including, for example, a title, description, origination location, sensitivity level, and the like. The user selection interface 112 may also provide functionality to identify and select a recipient user to receive the health data updates represented by the selected authorization identifiers 110. The user selection interface 112 may also include a share button 114. Selection of the share button 114 may cause the initiating user device 104(1) to generate the share request described herein based on the selected authorization identifiers 110 and send the share request to the recipient user device 104(2), as discussed next.

At block 116, the process 100 includes the recipient user device 104(2) receiving the sharing request from the initiating user device 104(1), and accepting the sharing request. Information about the sharing request may be used to populate an acceptance user interface 118 on the recipient user device 104(2). The acceptance user interface 118 may be used to present information about particular authorization identifiers 110(1) (and corresponding data types, including descriptions, examples, etc.) the initiating user intends to share with the recipient user. In this manner, the recipient user may decide whether they accept (or reject) the sharing request. The acceptance user interface 118 also includes an accept button 122 that may be used to accept the sharing request. In some examples, the acceptance user interface 118 may also include a reject button for rejecting the sharing request and/or a modify button for suggesting an alternate sharing request. For example, the recipient user may decide that they want to receive updates about a certain data type not represented by the particular authorization identifiers 110(1). The recipient user may use the acceptance user interface 118 to send a counter-request to the initiating user device 104(1). At the initiating user device 104(1), the initiating user may accept, reject, and/or suggest a modification to the counter-request. In some examples, the acceptance user interface 118 may provide functionality for the recipient user to send “share back” updates about the same or different data types.

At block 124, the process 100 includes the initiating user device 104(1) generating a transaction package based on the authorization identifiers 110(1). Block 124 may be performed after the initiating user device 104(1) has received confirmation of the recipient user device 104(2) having accepted the terms of the sharing request. Generating the transaction package may include the initiating user device 104(1) creating a summary of the health data updates as they are recorded. Each summary of a health data update may include a block of health data and metadata useable to decide how to present the block of health data. After the updates have been generated by the initiating user device 104(1), they may be bundled together into a single transaction package.

At block 126, the process 100 includes sending the transaction package to the service provider 106. In particular, the initiating user device 104(1) may send the transaction package, including the summaries of health data updates for the data types identified by the authorization identifiers, to the service provider 106. The service provider 106 may host a cloud-based storage service. For example, the transaction may be sent to the cloud-based storage service and stored, by the service provider 106, in a storage zone associated with an account profile of the initiating user. The transaction-based approach may be useful when the storage zone includes health-data updates for the initiating user collected and generated from different user devices (e.g., the initiating user device 104(1) and other user devices associated with the initiating user such as a watch, smartphone, tablet, laptop computer, etc.).

At block 128, the process 100 includes the recipient user device 104(2) requesting a transaction package from the service provider 106. As described herein, the recipient user device 104(2) may use a predefined routine to decide which transaction package to request from the service provider 206, as there may be multiple transaction packages available for some given time. For example, the recipient user device 104(2) may examine transaction metadata associated with each transaction package to determine whether one transaction package would be preferable to another. In some examples, the transaction metadata may include information such as, for example, a timestamp of the transaction generation, whether the transaction originated from the user device that is paired with a smartwatch, a static identifier of the user device used to generate the summaries, etc.

At block 130, the process 100 includes the recipient user device 104(2) extracting data from the transaction package to populate a user interface with health data updates. In some examples, the health data updates may be used to populate a user interface within an application (e.g., an application devoted to user health) or other suitable location. The health data updates may be used to trigger any suitable notification to alert the recipient user about new health updates. In some examples, certain health data updates may trigger more intrusive alerts. For example, if a health data update indicates that the initiating user might be having a cardiac episode, the health data update may be presented as a prominent pop-up type alert 132. On the other hand, if a health data update indicates that the initiating user has achieved a fitness goal (e.g., exercised for 30 minutes) for an entire week (e.g., a type of health summary or highlight 134), the health data update may be presented less prominently, or otherwise added to a dashboard/user interface without specifically notifying the recipient user. Other types of health data updates 136 may also be presented using the user interface, as notifications, or in any other suitable manner.

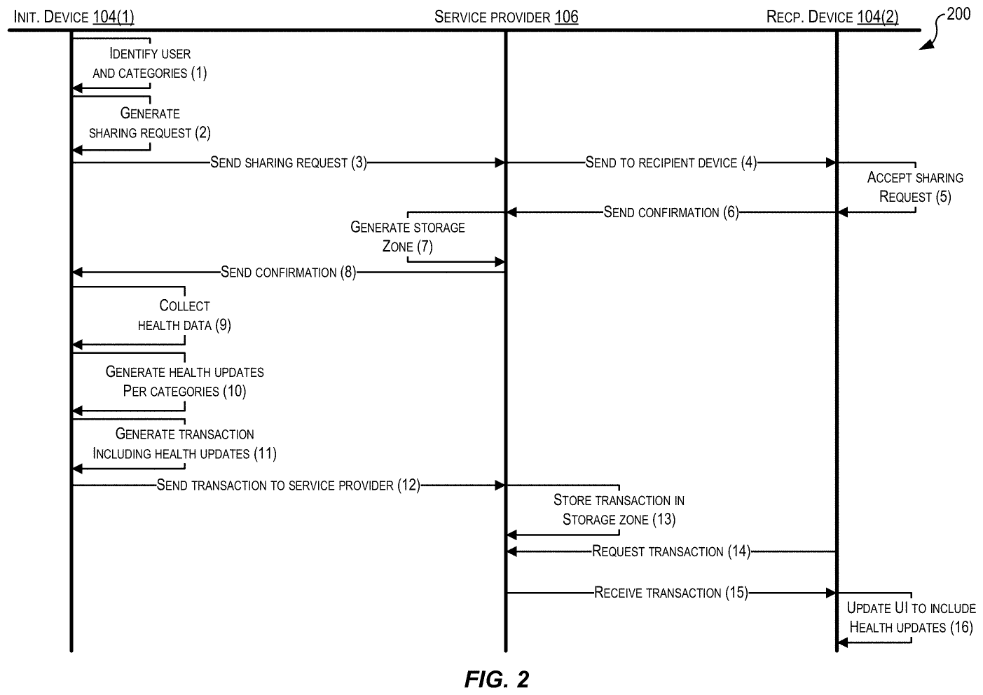

Turning now to FIG. 2, this figure illustrates a flowchart showing a process 200 for conducting sharing of health data updates among user devices, according to at least one example. The process 200 may be performed by the initiating user device 104(1), the recipient user device 104(2), and the service provider 106. The process 200 is a variation of the process 100, which includes various functions being performed by the various elements shown in FIG. 2 and described in additional detail in FIG. 1.

The process 200 may begin at (1), by the initiating user device 104(1) identifying a user and categories for sharing. The user may be associated with contact information on the initiating user device 104(1). In some examples, the user may be otherwise associated with a user of the initiating user device 104(1). For example, the user of (1) may be a social media contact of the user of the initiating user device 104(1). The categories may identify select categories and/or types of health data and may correspond to authorization identifiers. In some examples, (1) may be performed by the initiating user device 104(1) using an application resident on the initiating user device 104(1).

At (2), the process 200 includes the initiating user device 104(1) generating a sharing request. The sharing request may be based on the information collected at (1). For example, the sharing request may identify the user and which categories of health data the user of the initiating user device 104(1) intends to share with the user identified at (1).

At (3), the process 200 includes the initiating user device 104(1) sending the sharing request to the service provider 106. This may be performed using a messaging service, a notification service, or other suitable communication connection between the initiating user device 104(1) and the service provider 106. After receiving the sharing request, at (4), the process 200 includes the service provider 106 sending the sharing request to the recipient user device 104(2). In some examples, prior to sending to the recipient user device 104(2), the service provider 106 may log information from the sharing request, indicating that the user of the initiating user device 104(1) intends to share with the user of the recipient user device 104(2).

At (5), the process 200 includes the recipient user device 104(2) accepting the sharing request. This may include the user of the recipient user device 104(2) accepting the sharing request using a user interface presented at the recipient user device 104(2). In some examples, the user interface may enable the user of the recipient user device 104(2) to modify the sharing request and/or generate a comparable sharing request to be sent to the user of the initiating user device 104(1).

At (6), the process 200 includes the recipient user device 104(2) sending a confirmation to the service provider 106. Based on receiving the confirmation, the process 200 includes the service provider 106 generating a storage zone at (7). Generating the storage zone may be associated with the user of the initiating user device 104(1) and include a dedicated space for storing health data from the user of the initiating user device 104(1) that has been shared with the recipient user device 104(2). In some examples, the recipient user device 104(2) may also receive a confirmation that includes information about the storage zone (e.g., a storage zone uniform resource locator) and the like. In some examples, writing and reading data from the storage zone and the like may be implemented using existing data synching services. For example, each user of the recipient user device 104(2) and initiating user device 104(1) may have access to a cloud-based storage system, which, among other things, may be used to back up data from the recipient user device 104(2) and initiating user device 104(1). This cloud-based storage may also be leveraged for sharing health data, as described herein.

At (8), the process 200 includes the service provider 106 sending a confirmation to the initiating user device 104(1). The confirmation may identify that the recipient user device 104(2) has accepted the sharing request. In some examples, the confirmation may also include information about the storage zone (e.g., a URL or other resource locator) that is useable by the initiating user device 104(1) to access the storage zone.

At (9), the process 200 includes the initiating user device 104(1) collecting health data. This may include using sensors of the initiating user device 104(1) to collect health data, generating health data based on inputs from the user of the initiating user device 104(1), obtaining health data from one or more external sources such as from electronic medical record (EMR) systems configured to store the user's personal health record, obtaining health data from an accessory device such as a watch or other accessory sensor, and the like. The EMRs may store different versions of the user's personal health record, which may be processed on the initiating user device 104(1) and insights from that processing may constitute “collected health data.”

At (10), the process 200 includes the initiating user device 104(1) generating health updates per the categories. The health data updates may be generated according to the categories identified in the sharing request. In some examples, the health data updates may include health data updates for other categories as they relate to other sharing requests accepted by other users. The health updates may include content (e.g., a summary of the health data update) and metadata to present the content. The recipient user device 104(2) may use the metadata to decide how to present the summaries.

At (11), the process 200 includes the initiating user device 104(1) generating a transaction including the health updates. These may be the health updates generated at (10). The transaction may include multiple health updates bundled together in a single transaction. In some examples, a transaction is generated periodically, responsive to certain triggers (e.g., as new data comes in, as goals/thresholds are met, etc.), and in any other suitable manner. For example, logic may be configured to look for updates and generate transactions every hour, every few hours, every day, every few days, every week, etc. Generating the transaction may include generating transaction metadata that describes aspects of the transaction. For example, such metadata may include a timestamp, a version of the application used to generate the updates, whether the initiating user device 104(1) is paired with an accessory device, a source identifier for the initiating user device 104(1), and any other suitable information.

At (12), the process 200 includes the initiating user device 104(1) sending the transaction to the service provider 106. At (13), the process 200 includes the service provider 106 storing the transaction in the storage zone created previously. In some examples, at (12), the initiating user device 104(1) may also send a notification to the recipient user device 104(2) about the transaction. In other examples, the initiating user device 104(1) may not inform the recipient user device 104(2) about new transactions; rather, the recipient user device 104(2) may check the storage zone for updates.

At (14), the process 200 includes the recipient user device 104(2) requesting a transaction. This may include the recipient user device 104(2) requesting any new transactions stored in the storage zone. In some examples, the recipient user device 104(2) may read the transaction metadata to determine which transaction, among multiple transactions, to download. There may be multiple transactions because other user devices associated with the user of the initiating user device 104(1) may also upload transactions including health data. The recipient user device 104(2) may discriminate between these transactions to ensure a consistent and functional experience for users with multiple user devices.

At (15), the process 200 includes the recipient user device 104(2) receiving the transaction. This may include the service provider 106 sending the selected transaction to the recipient user device 104(2).

At (16), the process 200 includes the recipient user device 104(2) updating a user interface to include the health updates from the transaction. This may include the recipient user device 104(2) extracting the health updates from the transaction, including the summaries and metadata. The metadata may then be used by the recipient user device 104(2) to present the summaries.

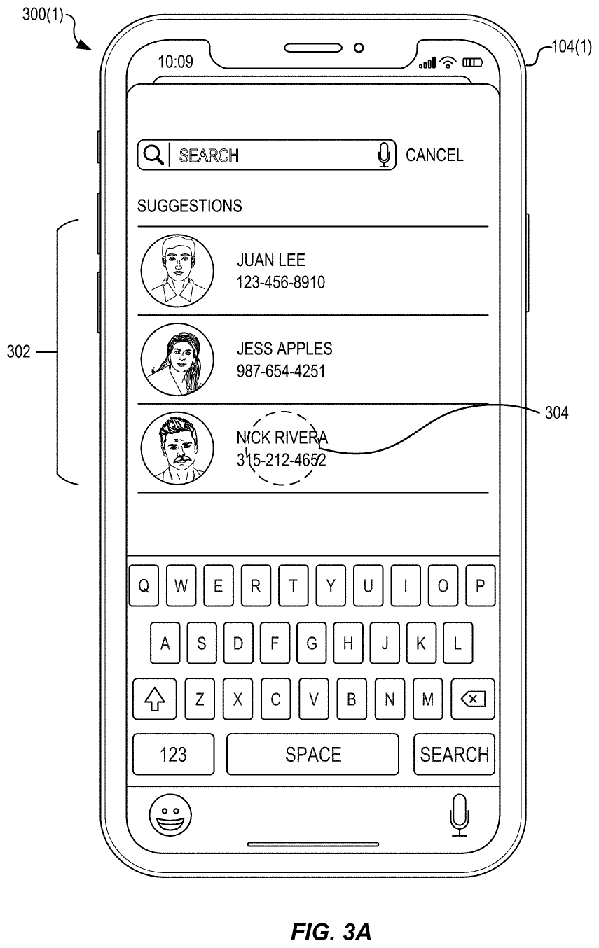

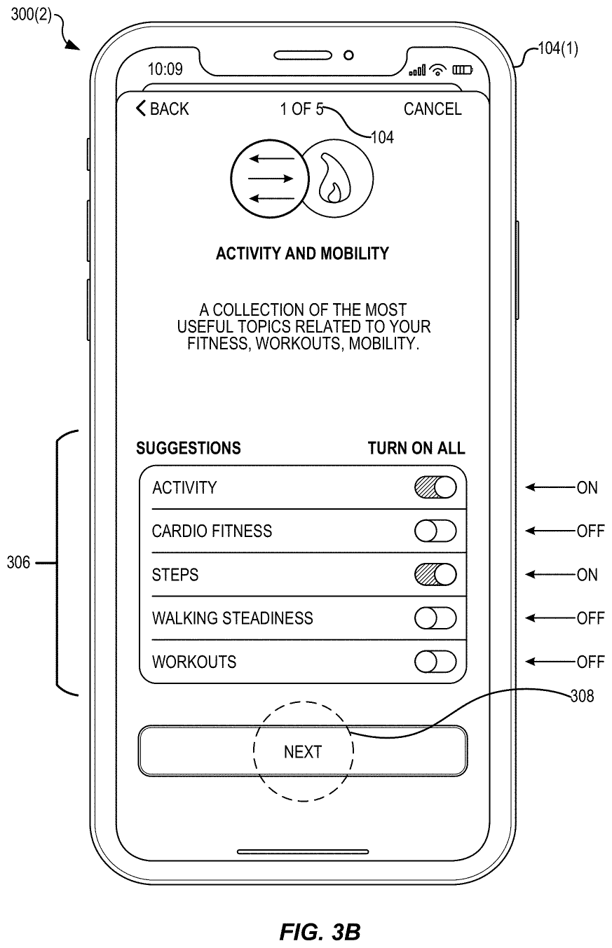

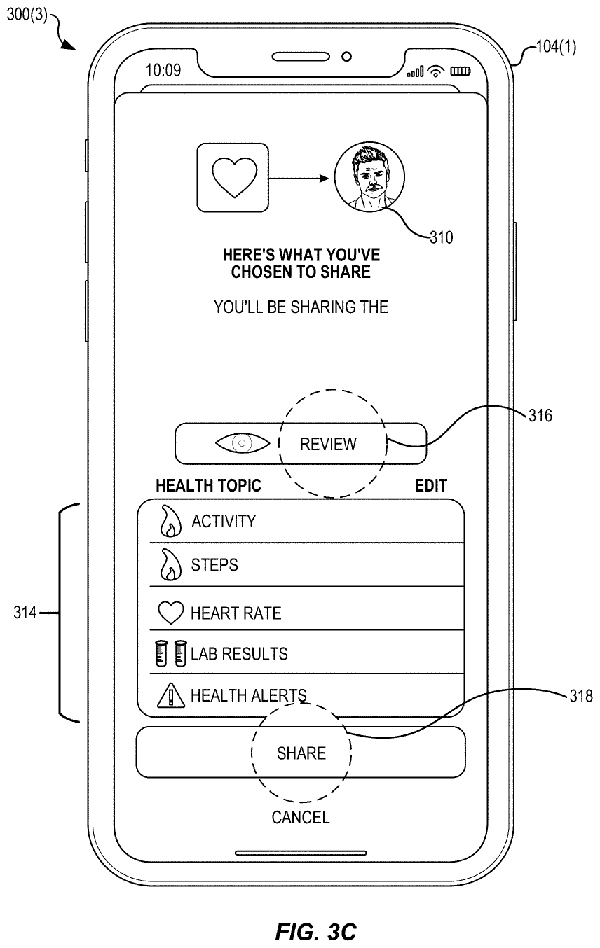

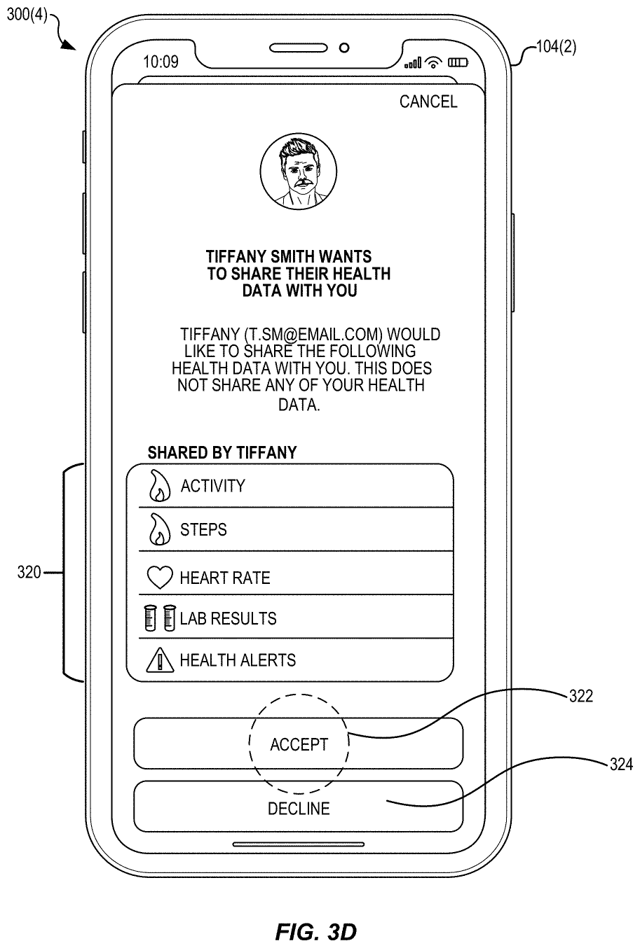

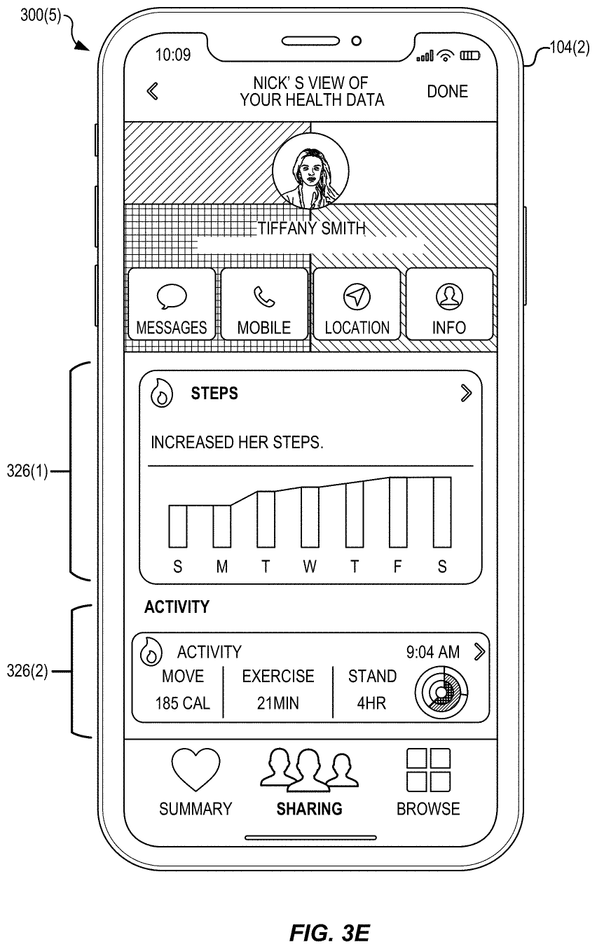

Turning now to FIGS. 3A-3E, these figures illustrate example user interfaces 300(1)-300(5) presented on a user device 104 for conducting sharing of health data updates among user devices, according to at least one example. In some examples, the user interfaces 300(1) to 300(5) may be presented on the recipient user device 104(2) or the initiating user device 104(1), depending on the user case. In FIGS. 3A-3E, the user interfaces 300(1), 300(2), 300(3), and 300(4) may be presented on the initiating user device 104(1), while the user interface 300(5) may be presented on the recipient user device 104(2). The user interfaces 300(1) to 300(5) may be presented using an application on the user device 104.

FIG. 3A depicts the user interface 300(1) on the initiating user device 104(1). The user interface 300(1) may be referred to a user selection user interface because this user interface may enable a user of the initiating user device 104(1) to select a user from a user list 302 with whom to share health data updates, as described herein. In some examples, the user interface 300(1) may enable the user of the initiating user device 104(1) to search for users using a search bar and input elements (e.g., keyboard, voice input, and the like). In some examples, the user list 302 may be prepopulated with suggested users. The suggested users may be identified by the initiating user device 104(1) using any appropriate technique. For example, the suggested users may be users that are identified as part of the same family as the user, are in the user's contacts, are caregivers, or are otherwise likely to accept a sharing request from the user of the initiating user device 104(1). As illustrated, the user of the initiating user device 104(1) has selected a user identified by “Nick Rivera,” depicted by an image, and having an associated phone number. Any of this information may constitute a user identifier (e.g., identifying information useable to generate a sharing request). In the illustrated version, the user of the initiating user device 104(1) has selected user “Nick Rivera,” as shown by selector 304. This is the user with whom the health updates will be offered for sharing.

FIG. 3B depicts the user interface 300(2) on the initiating user device 104(1). The user interface 300(2) may be referred to as an authorization identifier user interface because this user interface may enable the user of the initiating user device 104(1) to identify a set of authorization identifiers 306 to control which health data updates will be shared with the recipient user device 104(2). In some examples, an additional user interface may be presented that includes an option for the user of the initiating user device 104(1) to see a list of suggested topics that may be shared and to see all topics that may be shared. In some examples, other user interfaces may be presented to identify other sets of authorization identifiers. For example, the user interface 300(2) may be directed to “activity and mobility” type health data updates (e.g., fitness, workouts, mobility, and other similar types of health data topics), which may be one of many user authorization identifier user interfaces (e.g., the user interface 300(2) is “1 of 5” such interfaces). Other user authorization identifier user interfaces may include those directed to “basic health measurements” (e.g., weight, blood oxygen, respiratory rate, blood glucose, insulin, and other similar health data topics), “heart health” (e.g., resting heart rate, heart rate variability, heart rate, electrocardiograms, and other similar health data topics), “cycle tracking” (e.g., period predictions, fertility predictions, menstruation, sexual activity, and other similar health data topics), “lab results” (e.g., a list of lab results obtained from the user's health record, which may be selected one by one or by all and may include an option to share future updates), “heart alerts” (e.g., irregular heart rhythm, low blood pressure, and other similar health data topics), and any other type of health data update. As shown in FIG. 3B, the set of authorization identifiers 306 may include a set of selector type user interface elements each corresponding to one of the activity and mobility health data topics (e.g., activity, cardio fitness, steps, walking steadiness, workouts, and other similar health data topics). In this example, the user has selected the “activity” and “steps” selectors. In this manner, the user may intend to share updates about these health data types. Once the set of selectors has been selected, the user may select next button 308. As described above, this may cause the initiating user device 104(1) to present one of the other user authorization identifier user interfaces to allow the user to select other health data types for sharing. Once all selections have been made, the initiating user device 104(1) may present the user interface 300(3).

FIG. 3C depicts the user interface 300(3) on the initiating user device 104(1). The user interface 300(3) may be referred to as a sharing request user interface because this user interface may enable the user of the initiating user device 104(1) to view what health data types will be shared and with whom. In this example, the user interface 300(3) identifies the user “Nick Rivera” 310 and a summary of health data types 314, each of which has been selected using an authorization identifier selector. The user of the initiating user device 104(1) may select a review button 316 to review what health data updates will be shared. Once the user is satisfied with what health data will be shared and with whom, the user may select share button 318. This may cause the initiating user device 104(1) to generate a sharing request to be sent to the recipient user device 104(2) (e.g., Nick Rivera's user device) for further review by the user of the recipient user device 104(2).

FIG. 3D depicts the user interface 300(4) on the recipient user device 104(2). The user interface 300(4) may be referred to as an acceptance user interface because this user interface may enable the user of the recipient user device 104(2) (e.g., Nick Rivera) to accept or decline the sharing request initiated by the user of the initiating user device 104(1) (e.g., Tiffany Smith). The user interface 300(4) may include a summarized list 320 of what health data updates Tiffany intends to share. The summarized list 320 may correspond to the summary of health data updates 314 presented in the user interface 300(3) on the initiating user device 104(1). This may enable the user of the recipient user device 104(2) to review what information the user of the initiating user device 104(1) intends to share. If acceptable, the user of the recipient user device 104(2) may select an accept button 322. If the user of the recipient user device 104(2) does not want to receive the health data updates, they may select a decline button 324. In this example, the user of the recipient user device 104(2) has selected the accept button 322.

FIG. 3E depicts the user interface 300(5) on the recipient user device 104(2). The user interface 300(5) may be referred to as a health data summary user interface because this user interface may enable the user of the initiating user device 104(1) (e.g., Tiffany) to view a summary of what health data updates the user of the initiating user device 104(1) (e.g., Nick) will see. In particular, the user interface 300(5) identifies that the health data belongs to “Tiffany Smith” and includes information for communicating and/or otherwise interacting with this user. The user interface 300(5) also includes summaries 326(1) and 326(2), each of which may correspond to a type of health data update that has been shared with the user of the recipient user device 104(2). In this example, the summary 326(1) may correspond to the number of steps Tiffany has taken over the last week. In some examples, selection of the summary 326(1) may enable the user to view additional details about the particular type of health data update. The summary 326(2) may correspond to Tiffany's progress towards a set of health-related goals (e.g., a move goal, an exercise goal, and a stand goal). The summary 326(2) may also include a graphical indicator that corresponds to the set of health-related goals. In some examples, selection of the particular summary 326 may enable the user to view additional details about the particular type of health data update. The user interface 300(5) may be modified slightly and presented on the recipient user device 104(2). In particular, when health data updates are sent to the recipient user device 104(2), they may be in the form shown in the user interface 300(5).

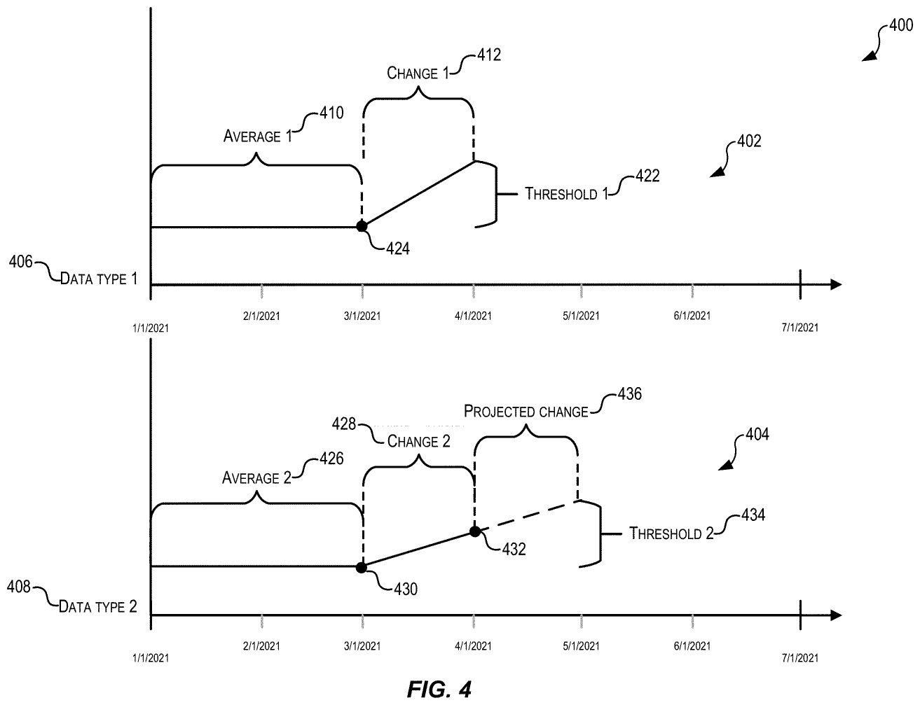

FIG. 4 illustrates a diagram 400 showing graphs 402 and 404 for identifying changes in health data and providing opportunistic notifications with user devices, according to at least one example. The diagram 400 is used for illustrative purposes to demonstrate techniques for identifying patterns in health data over time and for determining notifications relating to the same. For example, these patterns may indicate a trend in the health data (e.g., an inflection or change point that correlates to an increase in the measured data, a decrease, or even some indication of a steady state). The user device may be used to determine these trends based on actual health data and also determine whether to notify the user of the user device and/or users of devices with whom the user has shared their health data. For example, the initiating user device 104(1) may process its user's health data and decide whether to notify the user of the initiating user device 104(1) with information about the trend and may also send a notification to the recipient user device 104(2) (e.g., if a sharing relationship has been established, as described elsewhere herein). For data types that are correlated or otherwise associated, as described herein, the user device may determine whether to include an unripe notification together with a current ripe notification. In some examples, this determination may depend, at least in part, on whether the unripe notification is likely to become ripe within some period of time (e.g., the data indicates that the user is trending in a direction and is likely to continue). Such opportunistic determinations may reduce the overall number of notifications sent to the users, which may reduce alarm fatigue and improve user experience.

The graph 402 corresponds to a first data type 406 and includes a measurement of the data for the first data type 406 along the Y axis over a time period defined along the X axis. Similarly, the graph 404 corresponds to a second data type 408 and includes a measurement of the data for the second data type 408 along the Y axis over a time period defined along the X axis. The time periods in the graphs 402 and 404 may be the same. In some examples, the first and second data types 406 and 408 may be related in some manner. For example, the first data type 406 may represent user steps and the second data type 408 may represent workout minutes. Other example associations may include things like heart rate and workout, insulin level and burned calories, heart rate and steps, and any other association between at least two health data types. In some examples, the associations may include more than two health data types. The associations may be direct or inverse. For example, an increase in steps may be correlated with an increase in workout minutes, or a decrease in steps may be correlated with a higher resting heart rate. In any event, the techniques described herein may be used to make opportunistic notification determinations for correlated or otherwise related data types.

The graph 402 indicates that between Jan. 1, 2021 and Mar. 1, 2021, health data collected for the first data type 406 was relatively constant, as evidenced by a first average 410. However, between Mar. 1, 2021 and Apr. 1, 2021, the magnitude of the data collected began to increase, as evidenced by a first change 412. The techniques described herein may compare the first change 412 over the time period between March 1 and Apr. 1 to a first threshold 422 to determine whether a first change point 424 has been registered. The first threshold 422 may be specific to the first data type 406. If the increase in data has exceeded the first threshold 422 over the defined time period, the user device may determine the change point 424 (e.g., an inflection point at which the magnitude of the health data begins to increase). A change in the data like this over a period of time may be indicative of a trend. The user to whom the first data type 406 is associated may be interested to know about the trend. Thus, the user device may generate a notification to present to the user. In order to optimize resources and minimize overloading the user with notifications, the user device may also check whether any related data types are indicative of trends. The graph 404 is representative of such a check.

The graph 404 indicates that between Jan. 1, 2021 and Mar. 1, 2021, health data collected for the second data type 408 was relatively constant, as evidenced by a second average 426. However, between Mar. 1, 2021 and Apr. 1, 2021, the magnitude of the data collected began to increase, as evidenced by a second change 428. The techniques described herein may determine whether the second change 428 over the time period between points 430 and 432 is sufficient to exceed a second threshold 434. In this example, the second change 428 alone during these points does not meet or exceed the second threshold 434. However, the user device may continue to analyze the health data of the second data type to determine a projection 436. The projection 436 may represent a difference between the current average at 432 and the total threshold amount needed to qualify for a notification. If the user device determines with relative confidence that the user is likely to meet the second threshold 434 within some predefined amount of time beyond the current date (e.g., an hour, more than one hour, a day, more than one day, a week, more than one week, a month, more than one month, etc.), the user device may generate a notification about the trend for the second data type 408 and include it in the notification about the trend for the first data type 406.

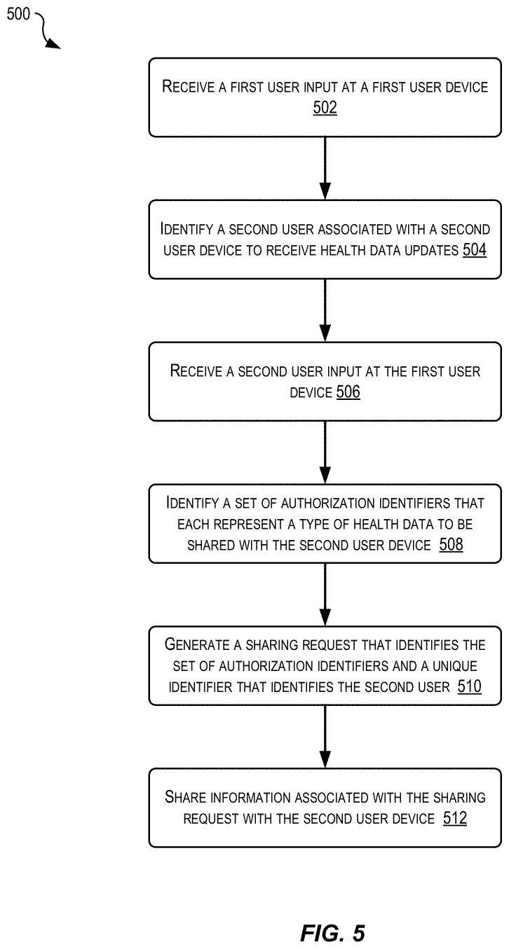

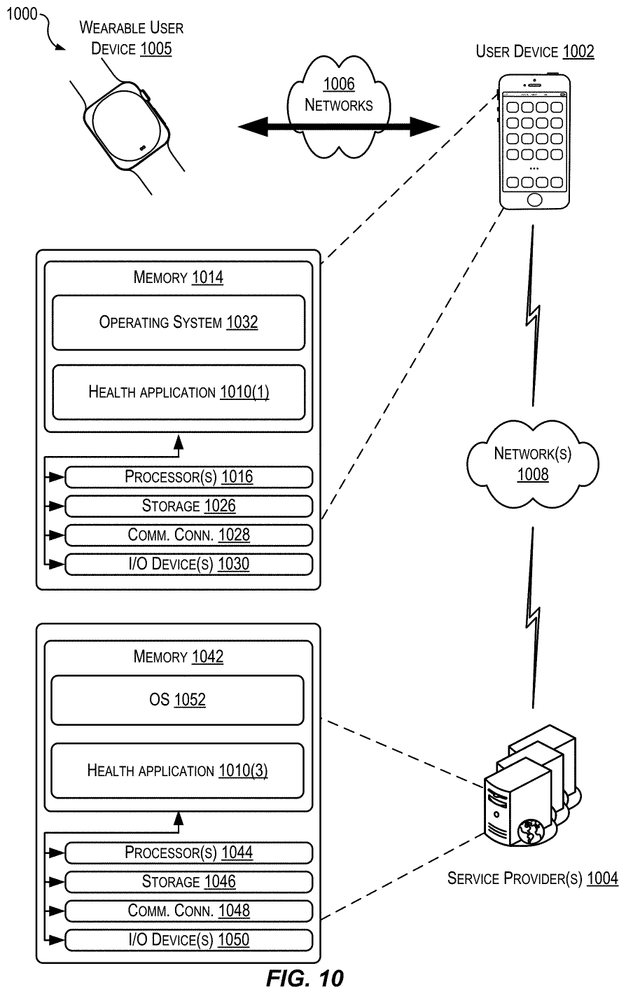

FIG. 5 illustrates a flowchart of a process 500 for conducting sharing of health data updates among user devices, according to at least one example. The process 500 may correspond in particular to an approach for initiating a sharing request for sharing health data updates with other associated users. A health application 1010 (FIG. 10), whether embodied in a wearable electronic device 1005 (FIG. 10), a user device 1002 (FIG. 10), or a service provider computer 1004 (FIG. 10), or any suitable combination of the foregoing may perform the process 500 of FIG. 5. Thus, while the process 500 is described as being performed by the user device, it should be understood that other user devices (e.g., the wearable user device) or a server may perform the process 500 with limited adjustments.

The process 500 begins at block 502 by a first user device receiving a first user input at the first user device. The first user input may be input by a first user and the first user input may identify a second user. In some examples, the first user input may be received at a user interface such as the user interface 300(1) (FIG. 3A). The first user device is an example of the initiating user device 104(1).

At block 504, the process 500 includes the first user device identifying the second user associated with a second user device to receive health data updates associated with the first user of the first user device. In some examples, the block 504 may be performed responsive to the block 502 being performed.

At block 506, the process 500 includes the first user device receiving a second user input at the first user device. The second user input may be input by the first user. In some examples, the second user input may be received at a user interface such as the user interface 300(2) (FIG. 3B).

At block 508, the process 500 includes the first user device identifying a set of authorization identifiers that each represent a type of health data update to be shared with the second user device. In some examples, the block 508 may be performed responsive to the block 506 being performed. The second user input may include a set of inputs to selectively identify individual authorization identifiers. In this manner, the first user may have a granular level of control over what type of health data updates may be shared with the second user. In some examples, individual authorization identifiers of the set of authorization identifiers may correspond to health topics. In some examples, the health data updates may be based on health data collected by or generated by at least one of the first user device or an accessory device of the first user device.

At block 510 the process 500 includes the first user device generating a sharing request that identifies the set of authorization identifiers and a unique identifier that identifies the second user. In some examples, the block 510 may be based at least in part on the information about the second user and the identified set of authorization identifiers. In some examples, the process 500 may further include presenting a summary of the set of authorization identifiers and the second user prior to or as part of generating the sharing request (e.g., the user interface 300(3) (FIG. 3C)). In some examples, the unique identifier may include a sharing identifier useable for uploading and downloading data from a server.

At block 512, the process 500 includes the first user device sharing information associated with the sharing request with the second user device. In some examples, sharing the information may be based on user input received at a user interface such as the user interface 300(3). In some examples, sharing the information associated with the sharing request with the second user device may include sending the information to a server. The information may be configured to enable the server to send the sharing request to the second user device. In some examples, the process 500 may further include receiving a message indicating acceptance of the sharing request by the second user, which may be proxied by the server or using other means.

In some examples, the process 500 may further include the first user device generating a set of health data updates based at least in part on the set of authorization identifiers, and combining the set of health data updates into a transaction. In some examples, these additional actions may be performed after the second user has accepted the sharing request. In some examples, each health update of the set of health updates may include source data and update metadata. In some examples, the transaction may include the set of health updates and transaction metadata. The update metadata may be useable by the second user device to determine display of the set of health updates, and the transaction metadata may be useable by the second user device to pick between different transactions.

In some examples, the process 500 may further include, prior to generating the set of health data updates, detecting, by the first user device, occurrence of a triggering event. The triggering event may include at least one of opening of a health application on the first user device, collection of a type of health data associated with the set of authorization identifiers, collection of a certain quantity of health data associated with the set of authorization identifiers, achievement of a health-based goal associated with the set of authorization identifiers, or a trend in health data associated with the set of authorization identifiers. In this manner, the first user device may check for updates when triggered.

In some examples, the process 500 may further include the first user device sending information associated with the transaction to a server to enable the server to store the transaction in accordance with a storage zone associated with the unique identifier that identifies the second user.

In some examples, the process 500 may further include the first user device generating a different sharing request that identifies a different set of authorization identifiers and a different unique identifier that identifies a third user of a third user device, and the first user device sending information associated with the different sharing request to a server. The information may be configured to enable the server to send the different sharing request to the third user device. In some examples, the different set of authorization identifiers may be distinct from the set of authorization identifiers.

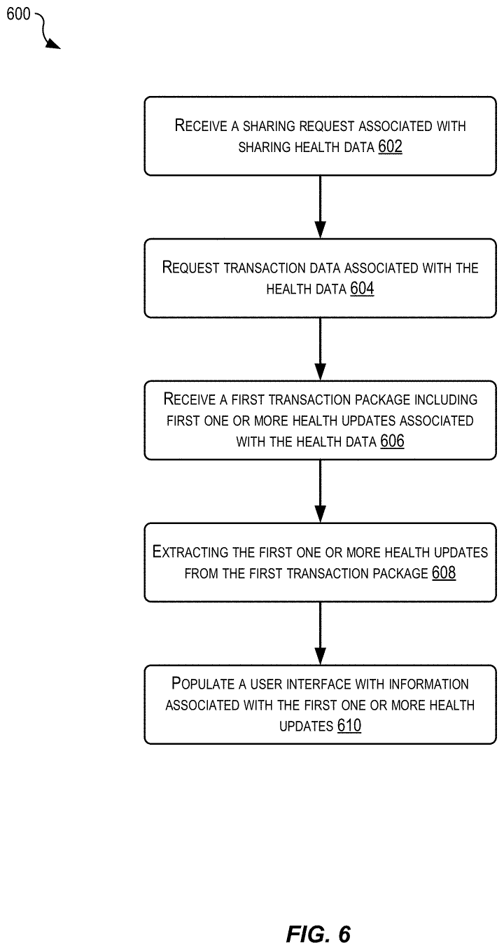

FIG. 6 illustrates a flowchart of a process 600 for conducting sharing of health data updates among user devices, according to at least one example. The process 600 may correspond in particular to an approach for receiving and accepting a sharing request for sharing health data updates with other associated users. The health application 1010 (FIG. 10), whether embodied in the wearable electronic device 1005 (FIG. 10), the user device 1002 (FIG. 10), or the service provider computer 1004 (FIG. 10), or any suitable combination of the foregoing may perform the process 600 of FIG. 6. Thus, while the process 600 is described as being performed by the user device, it should be understood that other user devices (e.g., the wearable user device) or a server may perform the process 600 with limited adjustments.

The process 600 begins at block 602 by a first user device receiving a sharing request associated with sharing health data. The first user device may be associated with a first user and the sharing request may be associated with sharing health data of a second user associated with a second user device with the first user device. In this example, the first user device is an example of the recipient user device 104(2) and the second device is an example of the initiating user device 104(1). In some examples, the request may include a unique identifier of the first user.

At block 604, the process 600 includes the first user device requesting transaction data associated with the health data. The transaction data may be stored by a server (e.g., the service provider computer 1004). In some examples, the process 600 may further include, prior to requesting the transaction, detecting, by the first user device, occurrence of a triggering event. The triggering event may include at least one of opening of a health application on the first user device, detecting that a time condition has been met, or the like. In some examples, the first user device may periodically request transaction data.

At block 606, the process 600 includes the first user device receiving, from the server, a first transaction package including first one or more health data updates associated with the health data of the second user. In some examples, the block 606 may be performed in response to requesting the transaction data by the first user device. In some examples, the server and/or the first user device may select the first transaction package from among a set of transaction packages stored by the server. In some examples, selecting one of the first transaction package or the second transaction package as the selected transaction package may be based at least in part on the set of transaction selection rules. This may include performing at least one of (i) determining collection time information for each of the first and second transaction packages based at least in part on transaction metadata associated respectively with each of the first and second transaction packages; (ii) determining application version information for each of the first and second transaction packages based at least in part on the transaction metadata respectively associated with each of the first and second transaction packages; (iii) determining accessory device information for each of the first and second transaction packages based at least in part on the transaction metadata respectively associated with each of the first and second transaction packages; or (iv) determining device identifier information associated with each of the second and third user devices used to collect the first and second transaction packages based at least in part on the transaction metadata respectively associated with each of the first and second transaction packages. In some examples, (i), (ii), (iii), and (iv) may represent the set of transaction selection rules, which may be prioritized. For example, where possible, the user device may first prefer a recently committed transaction (e.g., a transaction that was committed within the last 72 hours), second may prefer a transaction created on a newer version of the health application (e.g., as determined via a build tag of the health application), third may prefer a transaction created on a device with a paired wearable use device, and fourth may prefer a device with the lower static source identifier (e.g., prefer a newer device).

In some examples, a goal of this logic may be to provide a consistent and functional experience for users with multiple devices. In some examples, the logic may ensure a few outcomes such as preventing the initiating user device from jumping between different recipient user devices when more than one recipient user devices are used interchangeably. Additionally, this approach may prevent an old initiating user device from “blocking” newer data from appearing and allows transactions with new features (from new versions of software) to be prioritized where possible.

At block 608, the process 600 includes the first user device extracting the first one or more health data updates from the first transaction package. Extracting the first one or more health data updates may include receiving a notification about the health updates and requesting the specific data from the server. In some examples, the first user device receives the health updates including a blob of health data and metadata useable by the first user device for presenting the health updates.

At block 610, the process 600 includes the first user device populating a user interface of the first user device with information associated with the first one or more health data updates. This may be performed using metadata received with the first one or more health data updates and logic in the health application.

In some examples, the process 600 further includes, in response to requesting the transaction data, receiving, from the server, a second transaction package including second one or more health data updates associated with the health data of the second user associated with a third user device. In this example, the process 600 may further include selecting one of the first transaction package or the second transaction package as a selected transaction package based at least in part on a set of transaction selection rules. In this example, extracting the first one or more health data updates from the first transaction package may include extracting selected one or more health data updates from the selected transaction package. In this example, populating the user interface of the first user device with information associated with the first one or more health data updates may include populating the user interface of the first user device with information associated with the selected one or more health data updates.

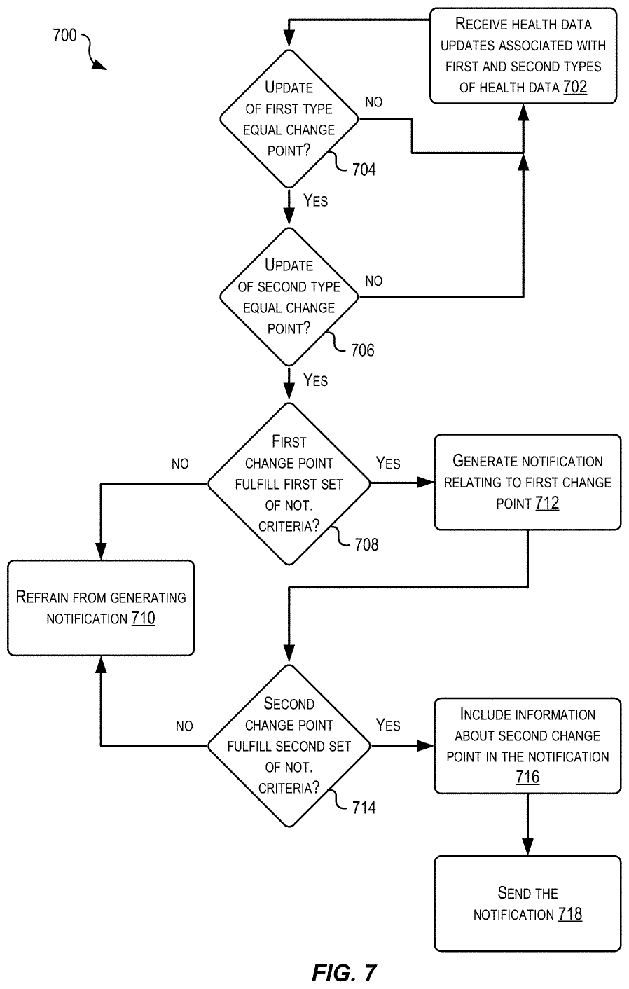

FIG. 7 illustrates a flowchart of a process 700 for identifying changes in health data and providing opportunistic notifications with user devices, according to at least one example. The process 700 may correspond in particular to an approach for a first user device to identify a trend in health data, decide whether to notify a user about the trend, and decide whether to notify the user about a trend in a related health data. The health application 1010 (FIG. 10), whether embodied in the wearable electronic device 1005 (FIG. 10), the user device 1002 (FIG. 10), or the service provider computer 1004 (FIG. 10), or any suitable combination of the foregoing may perform the process 700 of FIG. 7. Thus, while the process 700 is described as being performed by the user device, it should be understood that other user devices (e.g., the wearable user device) or a server may perform the process 700 with limited adjustments.

The process 700 begins at block 702 by a first user device receiving health data updates associated with first and second types of health data. The first user device may be associated with a first user. The health data updates may correspond to any of the type of health data described herein. For example, the health data updates may relate to data collected by the first user device, collected by an accessory user device, received from an electronic health record system, and the like. In some examples, the first health data of the first type of health data is collected by at least one of a handheld user device or a wearable user device.

At block 704, the process 700 includes the first user device determining whether an update of a first type of health data is equal to a change point. Determining whether the update constitutes a change point may include evaluating the health data in accordance with one or more change point criteria. For example, this may include evaluating the health data over a time window for significance, absolute change, and the like. This may include a predefined minimum P value for a change point. The time window may include multiple periods and the evaluation may include evaluating a current time period with respect to one or more other time periods. If the answer at block 704 is No, the process 700 returns to block 702 to receive additional health data updates. If the answer at block 704 is Yes, the process 700 continues to block 706. In some examples, the first change point may include a change in the first type of health data over a given time period.

In some examples, determining the first change point may include comparing first health data of the first type of health data from a first time period with second health data of the first type of health data from a second time period that overlaps with the first time period.

In some examples, the process 700 may include, after determining the first change point and before determining the second change point, picking the second type of health data to evaluate based at least in part on a predefined correlation between the first and second types of health data.

At block 706, the process 700 includes the first user device determining whether an update of a second type of health data is equal to a change point. This determination may be similar to the block 704. In some examples, the second type of health data may be correlated with the first type of health data. In some examples, the determination at 706 may be triggered by the determination at 704. In some examples, the standard for determining the second type of health data may be different at least because the determination at 706 is performed after the block 704, based on the specific type of health data, and based on other factors. For example, determining whether the update constitutes a change point may include evaluating the health data in accordance with one or more change point criteria. If the answer at block 706 is No, the process 700 returns to block 702 to receive additional health data updates. If the answer at block 706 is Yes, the process 700 continues to block 708.

In some examples, determining the first change point in the first type of health data may include determining the first change point in accordance with a first threshold, and determining the second change point in the second type of health data may include determining the second change point in accordance with a second threshold. The first threshold may be an absolute threshold and the second threshold is a relative threshold. In some examples, the relative threshold may depend at least in part on the first change point occurring prior to the second change point. In some examples, the first change point in the first type of health data may include determining the first change point at a first time. In some examples, determining the second change point in the second type of health data may include determining the second change at a second time following the first time.

In some examples, the first type of health data and the second type of health data may be associated with a single user profile and are indicative of health of a user associated with the single user profile.

At block 708, the process 700 includes the first user device determining whether the first change point fulfills a first set of notification criteria. This may include determining whether the first change point is sufficient for notifying the user. In some examples, a change point may include any inflection point in the data and the block 708 and 714 may be performed to determine whether the inflection point constitutes a sufficiently distinct change to notify the user. In some examples, the first change point may meet all notification criteria, e.g., the data indicates that a threshold has been exceeded over a time period indicating a significant change. If the answer at block 708 is No, the process 700 proceeds to block 710 and refrains from generating a notification. If the answer at block 708 is Yes, the process 700 proceeds to block 712.

In some examples, the first set of notification criteria may include a recency criterion defining a time threshold between sending notifications associated with the first type of health data, and a trend criterion defining conditions under which a change in direction of a trend in the first type of health data will trigger a notification.

In some examples, the determination that the first change points meets the first set of notification criteria may include determining, at a first time, that the first change point meets the first set of notification criteria. In some examples, the determination that the second change point meets the second set of notification criteria may include determining that the second change point is likely to occur within a threshold amount of time following the first time.

At block 712, the process 700 includes the first user device generating a notification relating to the first change point. The notification may identify the data type, the direction of the trend (e.g., up, down, etc.), and any other specifics about the trend (e.g., the time window over which the trend was evaluated, a suggestion for continuing the trend or reversing the trend, etc.). In some examples, the block 712 may include, in accordance with a determination that the first change point meets a first set of notification criteria, generating a notification that includes first information associated with the first change point.

At block 714, the process 700 includes the first user device determining whether the second change point fulfills a second set of notification criteria. In some examples, the block 714 may include, in accordance with a determination that a second change point meets a second set of notification criteria, second information associated with the second change point in the notification. The second information may relate to the second change point. In some examples, the block 714 may include determining whether the second change point is sufficient for notifying the user. In some examples, a change point may include any inflection point in the data and the block 708 and 714 may be performed to determine whether the inflection point constitutes a sufficiently distinct change to notify the user. In some examples, the second set of notification criteria may be related to the first set of notification criteria. For example, the second set of notification criteria may consider whether the second change point meets a threshold requirement or whether the threshold requirement is likely to be met within some given time period (e.g., within a short period of time). In this manner, the second set of notification criteria may consider whether the user should be notified about both the first and second change points, even if the first change point is the only one that is entirely ripe for notifying.

In some examples, the first set of notification criteria may be independent of the second type of health data and the second set of notification criteria may be dependent on the first type of health data.

If the answer at block 714 is No, the process 700 proceeds to block 710 and refrains from generating a notification. If the answer at block 714 is Yes, the process 700 proceeds to block 716.

At block 716, the process 700 includes the first user device including information about the second change in the notification. This may include adding information about a second trend in the notification.

At block 718, the process 700 includes sending the notification. This can include presenting the notification at the first user device and/or sending the notification to a second user device.

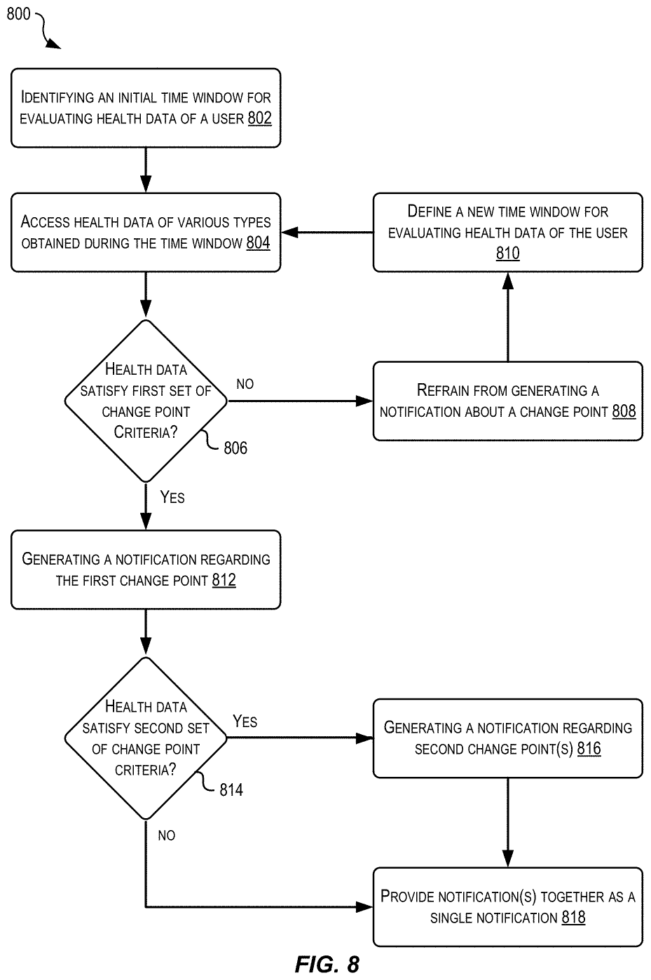

FIG. 8 illustrates a flowchart of a process 800 for identifying changes in health data and providing opportunistic notifications with user devices, according to at least one example. The process 800 may correspond in particular to an approach for a first user device to identify a trend in health data, decide whether to notify a user about the trend, and decide whether to notify the user about a trend in other health data. The health application 1010 (FIG. 10), whether embodied in the wearable electronic device 1005 (FIG. 10), the user device 1002 (FIG. 10), or the service provider computer 1004 (FIG. 10), or any suitable combination of the foregoing may perform the process 800 of FIG. 8. Thus, while the process 800 is described as being performed by the user device, it should be understood that other user devices (e.g., the wearable user device) or a server may perform the process 800 with limited adjustments.

The process 800 begins at block 802 by a user device identifying an initial time window for evaluating health data of a user. The initial time window may be predefined by the user device and/or by a service provider such as the service provider computer 1004. In some examples, the time window may correspond to a period of time over which health data of the user will be evaluated. For example, the time window may correspond to one month, two months, three months, four months, five months, six months, seven months, eight months, twelve months, two years, and any other suitable period. In some examples, the time window may be of suitable length that actual insights in the from of data trends may be identified. For example, if the time window is too short, the amount of data collected may be too small to identify a trend with any suitable degree of certainty. In some examples, more than one time window may be used. For example, the process 800 may be performed on a short time window (e.g., four weeks) and repeated on a long time window (e.g., twenty six weeks). In some examples, certain data types may be evaluated in one window, but not the other. In some examples, all data types may be evaluated in both windows.

At block 804, the process 800 includes the user device accessing health data of various types obtained during the time window. The health data may be accessed from a database on the user device that stores the health data. The health data may be collected by the user device, received from a different user device and/or accessory device, and collected using any other suitable method (e.g., from an electronic health record (EHR) computer system associated with the user). In some examples, the health data may be organized by data type such that each data type may be evaluated independently of other data types. Thus, accessing the health data of various types may include individually and independently accessing health data of various types from one or more sources. In some examples, the user device may process the health data to some extent after accessing the health data. For example, this may include aligning, normalizing, removing outliers, and performing any other data rectification post-processing action.

At block 806, the process 800 includes the user device determining whether the health data satisfies a first set of change point criteria. In some examples, the set of change point criteria may correspond to a set of statistical measures, to which thresholds may be applied to sort and compare the health data of the same type. In some examples, the user device may use a dynamically adjustable window to evaluate different sub portions of the health data within the larger time window. For example, continuing with the example from above, the time window may be around four weeks and the dynamically adjustable time window may be set initially at five days, but may then be increased, decreased, and/or moved throughout the time window to evaluate different portions of the health data that may satisfy the first set of change point criteria. As part of determining whether the health data satisfies the first set of change point criteria, the user device may evaluate the health data in the dynamically adjustable time window with the goal of dividing the window into a pre-change point sub-window and a post-change point sub-window, with the change point being located at a time between the pre- and post-change point sub-windows. The first set of change point criteria may indicate what differences in the health data must be present in order to identify the change point between the two sub-windows of the time period.

In some examples, the first set of criteria may correspond to a Welch's t-test (or other similar test such as Student's t-test) that is indicative of statistical significance between two sets of data (e.g., a pre-change point sub-window and a post-change point sub-window), an effect size determination that is indicative of the magnitude of relative change between the data in the two sub-windows, and any other statistical or other criteria for assessing whether the user's health data is indicative of a consistent change in one direction or the other. In some examples, the first set of change point criteria may represent a stringent test for determining whether a change point is present in the health data. In other words, to satisfy the first set of change point criteria the difference in the health data may have to be larger and/or be present for a more significant period of time, as compared to other sets of change point criteria described herein (e.g., a second set of change point criteria of block 814). As introduced herein, the first set of change point criteria may also include a minimum threshold, which may represent an amount of change in the data needed in order to be medically relevant. The minimum thresholds may be determined by medical professionals and/or using modeling of prior use cases. Each data type may be associated with its own minimum threshold, which may also depend on the length of the time period under consideration and whether the data is being evaluated as an initial change point (e.g., possibly a higher threshold) or a second change point (e.g., possibly a lower threshold). The minimum thresholds may be hardcoded.

If the answer at block 806 is No, the process 800 continues to block 808. At block 808, the process 800 includes the user device refraining from generating a notification about a change point. Block 808 may essentially mean that because a change point was not identified at block 804, the user device does not need to generate a notification.

At block 810, the process 800 includes the user device defining a new time window for evaluating health data of the user. The new time window may be a new dynamically adjustable time window, as described herein. In some examples, the time window may be a new time window, as compared to the initial time window. For example, the initial time window may be four weeks and the new time window may be twenty-six weeks. Following block 810, the process 800 includes the user device accessing the health data of various types obtained during the time window, e.g., the new window this time. The loop 804, 806, 808, 810, and back to 804 may repeat iteratively until all of the health data of the user (e.g., different data types) has been evaluated across different windows using the first set of change point criteria. If during any of these iterative loops, an answer at 806 is Yes, the process 800 proceeds to block 812.

At block 812, the process 800 includes the user device generating a notification regarding the first change point. The notification may include information about the change point such as a textual description of how the data changed, a date on which the change point occurred, a graphical depiction of the data corresponding to the change point, and any other information about the change point. The notification may include a payload of data and metadata to enable appropriate rendering of the notification on a display screen of the user device.

At block 814, the process 800 includes the user device determining whether the health data satisfies a second set of change point criteria. In some examples, the second set of change point criteria may correspond to the first set of change point criteria, but be distinct from the first set of change point criteria in terms of strictness. For example, the second set of change point criteria may represent criteria that are relaxed with respect to the first set of change point criteria. In other words, health data that meets the first set of change point criteria will always meet the second change point criteria, but health data that meets the second set of change point criteria may not also meet the first set of change point criteria. When health data of a first data type has been found to satisfy the first set of change point criteria, the evaluation at block 814 may be of health data of other data types that might be correlated with the first data type. For example, assuming that the evaluation at block 806 indicated that user has increased her step count such that the user device has identified a change point in the step data type, block 814 may look at all other data types to identify if any other data types are also trending upwards (e.g., workout minutes, etc.), even if the other data types are lagging behind the step data. This is why the second set of change point criteria may have lower thresholds as compared to the first set of change point criteria.

In practice, the evaluation at block 814 will look for change points under a less strict standard, as compared to the first set of change point criteria, in order to then generate a notification about these change points to include with the notification generated at 812. Thus, if the answer at block 814 is Yes, the process 800 proceeds to block 816. At block 816, the process 800 includes the user device generating a notification regarding the second change point(s). These notifications may represent data types, for which the second set of change point criteria have been satisfied. The notifications may be combined with the notification generated at block 812 and provided, by the user device, for presentation at the user device (e.g., a display screen) as a single notification at block 818. If the answer at block 814 is No, the process 800 continues directly to block 818 and delivers the notification regarding the first change point generated at 812 without including any other notifications regarding any other change points together with the notification from 812.

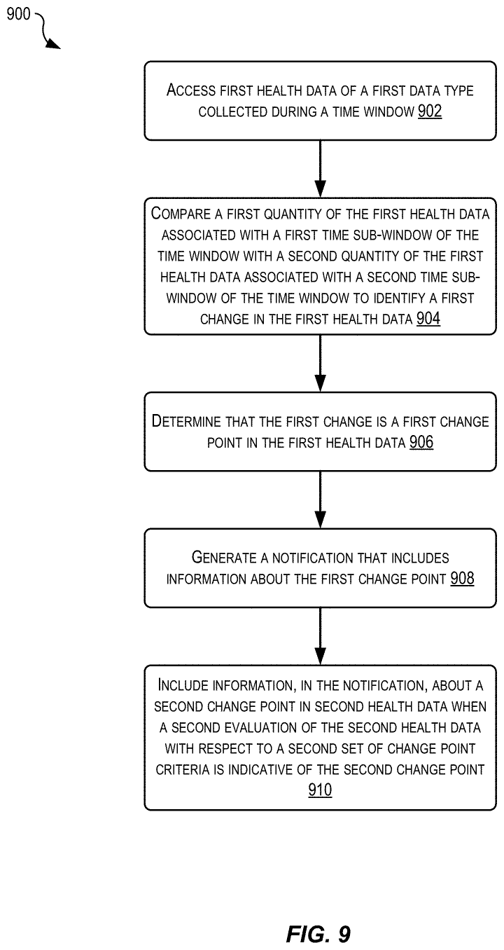

FIG. 9 illustrates a flowchart of a process 900 for identifying changes in health data and providing opportunistic notifications with user devices, according to at least one example. The process 900 may correspond in particular to an approach for a first user device to identify a trend in health data, decide whether to notify a user about the trend, and decide whether to notify the user about a trend in other health data. The health application 1010 (FIG. 10), whether embodied in the wearable electronic device 1005 (FIG. 10), the user device 1002 (FIG. 10), or the service provider computer 1004 (FIG. 10), or any suitable combination of the foregoing may perform the process 900 of FIG. 9. Thus, while the process 900 is described as being performed by the user device, it should be understood that other user devices (e.g., the wearable user device) or a server may perform the process 900 with limited adjustments.

The process 900 begins at block 902 by a user device accessing first health data of a first data type collected during a time window. In some examples, the time window may be at least one of about one month or about six months. In some examples, the first health data and the second health data may collected by one or more sensors of the user device, by one or more sensors of a different user device and shared with the user device, by one or more sensors of an accessory user device and shared with the user device, accessed from memory such as a database on the user device, accessed from a cloud-based storage service, and obtained in any other suitable manner.

At block 904, the process 900 includes the user device comparing a first quantity of the first health data associated with a first time sub-window of the time window with a second quantity of the first health data associated with a second time sub-window of the time window to identify a first change in the first health data.

In some examples, the process 900 may further include the user device iteratively evaluating health data from differently-sized sub-windows within the time window to define the first time sub-window and the second time sub-window.

At block 906, the process 900 includes the user device determining that the first change is a first change point in the first health data. This may include determining in accordance with a first evaluation of the first and second quantities of the first health data with respect to a first set of change point criteria. In some examples, the first change point may include a data point in the first time window when the first health data is indicative of an upward trend or a downward trend.

At block 908, the process 900 includes the user device generating a notification that includes information about the first change point.

At block 910, the process 900 includes the user device including information, in the notification, about a second change point in second health data when a second evaluation of the second health data with respect to a second set of change point criteria is indicative of the second change point. In some examples, the first change point may be statistically correlated with the second change point. In some examples, the notification may include one or more graphical elements corresponding to at least one of the first change point or the second change point. In some examples, the first and second health data may be associated with a user. In this example, the first change point and the second change point may be related to a behavior of the user relating to health.

In some examples, the second health data may be of a second data type that is distinct from the first data type and are collected during the time window. In some examples, the first set of change point criteria may include a first set of thresholds and the second set of change point criteria may include a second set of thresholds that is distinct from the first set of thresholds. In some examples, the second set of thresholds may be relaxed with respect to the first set of thresholds. In some examples, the first set of change point criteria may include a first statistical significance criterion (e.g., a first P value determined using a T test) and a first statistical magnitude criterion (e.g., a first effect size value), and the second set of change point criteria may include a second statistical significance criterion (e.g., a second P value determined using a T test) and a second statistical magnitude criterion (e.g., a second effect size value). In some examples, the statistical significance criteria may be different between the first and second sets, and the statistical magnitude criteria may be the same between the first and second sets. As introduced herein, the second set of change point criteria may also include a minimum threshold, which may represent an amount of change in the data needed in order to be medically relevant.

In some examples, a first change point criterion of the first set of change point criteria is unique to the first data type and a second change point criterion of the first set of change point criteria is common with a corresponding third change point criterion of the second set of change point criteria.

In some examples, the process 900 may further include the user device providing the notification for presentation at a display of a user device. In some examples, the process 900 may further include refraining from generating additional notifications for a fixed period time following providing the notification for presentation at the display of the user device. This fixed time period may correspond to a cool down period in which the user device refrains from notifying the user about the changes in the data of the particular data type. This may be used to ensure that the user does not get too many notifications, especially when they are changing their behavior in a sufficiently consistent and significant manner that notifications could be triggered more frequently (e.g., increased their number of daily steps by 1000 each day over a week).

In some examples, the process 900 may further include the user device comparing a third quantity of the first health data associated with a third time sub-window of the time window with a fourth quantity of the first health data associated with a fourth time sub-window of the time window to identify a third change in the first health data. In this example, the process 900 may further include the user device, in accordance with a third evaluation of the third and fourth quantities of the first health data with respect to the first set of change point criteria, determining that the third change is a third change point in the first health data. In this example, the process 900 may further include the user device selecting between the first change point and the third change point based on respective magnitudes of the first change and the third change. For example, as both the first and third change points may indicate the user has experienced statistically significant changes in behavior during the time window, the user device may select the change point with the larger magnitude as the selected change point for purposes of notifying the user. In this example, generating the notification may include generating the notification that includes information about the selected change point.

In some examples, using the health update sharing techniques described herein, the first health data may be shared with a different user device for processing. For example, the process 900 may include a first user device associated with a first user collecting the first health data. In this example, accessing the first health data at block 902 may include receiving, by a second user device associated with a second user, the first health data from the first user device. In this example, the process 900 may further include providing the notification for presentation at the second user device. In this example, the process 900 may further include the second user device requesting the first health data from a cloud-based server that stores the first health data on behalf of the first user.

FIG. 10 illustrates an example architecture or environment 1000 configured to implement techniques relating to conducting sharing of health data updates among user devices, according to at least one example. In some examples, the example architecture 1000 may further be configured to enable a user device 1002 (e.g., the user device 104), the service provider computers 1004 (e.g., the service provider 106), and a wearable electronic device 1005 (e.g., an example accessory deice) to share information. In some examples, the devices may be connected via one or more networks 1008 and/or 1006 (e.g., via Bluetooth, WiFi, the Internet, or the like). In the architecture 1000, one or more users may utilize the user device 1002 to manage, control, or otherwise utilize the wearable electronic device 1005, via the one or more networks 1006. Additionally, in some examples, the wearable electronic device 1005, the service provider computers 1004, and the user device 1002 may be configured or otherwise built as a single device. For example, the wearable electronic device 1005 and/or the user device 1002 may be configured to implement the examples described herein as a single computing unit, exercising the examples described above and below without the need for the other devices described.

In some examples, the networks 1006, 1008 may include any one or a combination of many different types of networks, such as cable networks, the Internet, wireless networks, cellular networks, satellite networks, other private and/or public networks, or any combination thereof. While the illustrated example represents the user device 1002 accessing the service provider computers 1004 via the networks 1008, the described techniques may equally apply in instances where the user device 1002 interacts with the service provider computers 1004 over a landline phone, via a kiosk, or in any other manner. It is also noted that the described techniques may apply in other client/server arrangements (e.g., set-top boxes, etc.), as well as in non-client/server arrangements (e.g., locally stored applications, peer to peer configurations, etc.).

As noted above, the user device 1002 may be configured to collect and/or manage user activity data potentially received from the wearable electronic device 1005. In some examples, the wearable electronic device 1005 may be configured to provide health, fitness, activity, and/or medical data of the user to a third- or first-party application (e.g., the service provider computer 1004). In turn, this data may be used by the user device 1002 to identify trends and/or for sharing. The user device 1002 may be any type of computing device such as, but not limited to, a mobile phone, a smartphone, a personal digital assistant (PDA), a laptop computer, a desktop computer, a thin-client device, a tablet computer, a wearable device, or the like. In some examples, the user device 1002 may be in communication with the service provider computers 1004 and/or the wearable electronic device 1005 via the networks 1008, 1006, or via other network connections.

In one illustrative configuration, the user device 1002 may include at least one memory 1014 and one or more processing units (or processor(s)) 1016. The processor(s) 1016 may be implemented as appropriate in hardware, computer-executable instructions, firmware, or combinations thereof. Computer-executable instruction or firmware implementations of the processor(s) 1016 may include computer-executable or machine-executable instructions written in any suitable programming language to perform the various functions described. The user device 1002 may also include geo-location devices (e.g., a global positioning system (GPS) device or the like) for providing and/or recording geographic location information associated with the user device 1002. In some examples, the wearable user device 1005 may also include geo-location devices for providing and/or recording geographic location information associated with wearable user device 1005.

The memory 1014 may store program instructions that are loadable and executable on the processor(s) 1016, as well as data generated during the execution of these programs. Depending on the configuration and type of the user device 1002, the memory 1014 may be volatile (such as random access memory (RAM)) and/or non-volatile (such as read-only memory (ROM), flash memory, etc.). The user device 1002 may also include additional removable storage and/or non-removable storage 1026 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 1014 may include multiple different types of memory, such as static random access memory (SRAM), dynamic random access memory (DRAM), or ROM. While the volatile memory described herein may be referred to as RAM, any volatile memory that would not maintain data stored therein once unplugged from a host and/or power would be appropriate.

The memory 1014 and the additional storage 1026, both removable and non-removable, are all examples of non-transitory computer-readable storage media. For example, non-transitory computer readable storage media may include volatile or non-volatile, removable or non-removable media implemented in any method or technology for storage of information such as computer-readable instructions, data structures, program modules, or other data. The memory 1014 and the additional storage 1026 are both examples of non-transitory computer storage media. Additional types of computer storage media that may be present in the user device 104 may include, but are not limited to, phase-change RAM (PRAM), SRAM, DRAM, RAM, ROM, Electrically Erasable Programmable Read-Only Memory (EEPROM), flash memory or other memory technology, compact disc read-only memory (CD-ROM), digital video disc (DVD) or other optical storage, magnetic cassettes, magnetic tape, magnetic disk storage or other magnetic storage devices, or any other medium that can be used to store the desired information and that can be accessed by the user device 1002. Combinations of any of the above should also be included within the scope of non-transitory computer-readable storage media. Alternatively, computer-readable communication media may include computer-readable instructions, program modules, or other data transmitted within a data signal, such as a carrier wave, or other transmission. However, as used herein, computer-readable storage media does not include computer-readable communication media.

The user device 1002 may also contain communications connection(s) 1028 that allow the user device 1002 to communicate with a data store, another computing device or server, user terminals, and/or other devices via the networks 1008, 1006. The user device 1002 may also include I/O device(s) 1030, such as a keyboard, a mouse, a pen, a voice input device, a touch input device, a display, an operating system 1032 and/or one or more application programs or services for implementing the features disclosed herein including a health application 1010(1). In some examples, the health application 1010(1) may be configured to implement the features described herein. As described in detail with reference to later figures, the wearable user device 106 may include a memory that includes a similar health application 1010(2), which may be accessible by one or more processors of the wearable user device 106. The service provider computer 1004 may also include a memory 1042 that includes a health application 1010(3). In this manner, the techniques described herein may be implemented by any one, or a combination of more than one, of the computing devices (e.g., the wearable user device 1005, the user device 1002, or the service provider computer 1004).

The service provider computers 1004 may also be any type of computing device such as, but not limited to, a mobile phone, a smartphone, a PDA, a laptop computer, a desktop computer, a thin-client device, a tablet computer, a wearable device, a server computer, a virtual machine instance, etc. In some examples, the service provider computers 1004 may be in communication with the user device 1002 and/or the wearable user device 1005 via the networks 1008, 1006, or via other network connections.

In one illustrative configuration, the service provider computers 1004 may include at least one memory 1042 and one or more processing units (or processor(s)) 1044. The processor(s) 1044 may be implemented as appropriate in hardware, computer-executable instructions, firmware, or combinations thereof. Computer-executable instruction or firmware implementations of the processor(s) 1044 may include computer-executable or machine-executable instructions written in any suitable programming language to perform the various functions described.

The memory 1042 may store program instructions that are loadable and executable on the processor(s) 1044, as well as data generated during the execution of these programs. Depending on the configuration and type of service provider computer 1004, the memory 1042 may be volatile (such as RAM) and/or non-volatile (such as ROM, flash memory, etc.). The service provider computer 1004 may also include additional removable storage and/or non-removable storage 1046 including, but not limited to, magnetic storage, optical disks, and/or tape storage. The disk drives and their associated non-transitory computer-readable media may provide non-volatile storage of computer-readable instructions, data structures, program modules, and other data for the computing devices. In some implementations, the memory 1042 may include multiple different types of memory, such as SRAM, DRAM, or ROM. While the volatile memory described herein may be referred to as RAM, any volatile memory that would not maintain data stored therein once unplugged from a host and/or power would be appropriate. The memory 1042 and the additional storage 1046, both removable and non-removable, are both additional examples of non-transitory computer-readable storage media.

The service provider computer 1004 may also contain communications connection(s) 1048 that allow the service provider computer 1004 to communicate with a data store, another computing device or server, user terminals and/or other devices via the networks 1008, 1006. The service provider computer 1004 may also include I/O device(s) 1050, such as a keyboard, a mouse, a pen, a voice input device, a touch input device, a display, speakers, a printer, etc.

Turning to the contents of the memory 1042 in more detail, the memory 1042 may include an operating system 1052 and/or one or more application programs or services for implementing the features disclosed herein including the health application 1010(3).

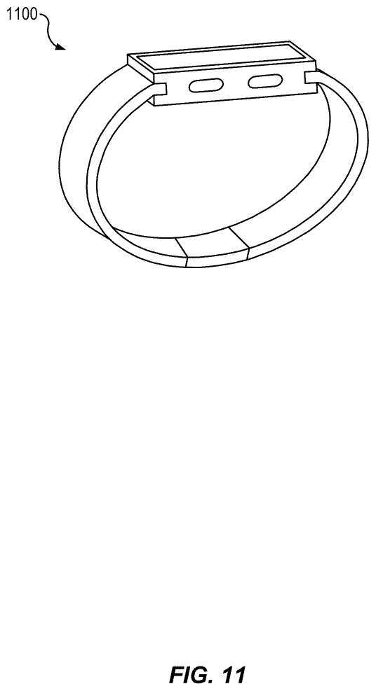

Examples described herein may take the form of, be incorporated in, or operate with a suitable wearable electronic device. One example of such a device is shown in FIG. 11 and takes the form of a wearable mechanism 1100. As shown, the mechanism 1100 may be worn on a user's wrist and secured thereto by a band. The mechanism 1100 may have a variety of functions including, but not limited to: keeping time; monitoring a user's physiological signals and providing health-related information based at least in part on those signals; communicating (in a wired or wireless fashion) with other electronic devices, which may be different types of devices having different functionalities; providing alerts to a user, which may include audio, haptic, visual and/or other sensory output, any or all of which may be synchronized with one another; visually depicting data on a display; gathering data from one or more sensors that may be used to initiate, control, or modify operations of the device; determining a location of a touch on a surface of the device and/or an amount of force exerted on the device, and using either or both as input; accepting voice input to control one or more functions; accepting tactile input to control one or more functions; and so on.

Alternative examples of suitable electronic devices include a phone; a tablet computing device; a portable media player; and so on. Still other suitable electronic devices may include laptop/notebook computers, personal digital assistants, touch screens, input-sensitive pads or surfaces, and so on.

In some examples the electronic device may accept a variety of bands, straps, or other retention mechanisms (collectively, “bands”). These bands may be removably connected to the electronic device by a lug that is accepted in a recess or other aperture within the device and locks thereto. The lug may be part of the band or may be separable (and/or separate) from the band. Generally, the lug may lock into the electronic device's recess and thereby maintain connection between the band and device. The user may release a locking mechanism to permit the lug to slide or otherwise move out of the recess. In some examples, the recess may be formed in the band and the lug may be affixed or incorporated into the device.

A user may change combinations of bands and electronic devices, thereby permitting mixing and matching of the two categories. It should be appreciated that devices having other forms and/or functions may include similar recesses and may releasably mate with a lug and/or band incorporating a lug. In this fashion, an ecosystem of bands and devices may be envisioned, each of which is compatible with another. A single band may be used to connect to devices, as one further example; in such examples the band may include electrical interconnections that permit the two devices to transmit signals to one another and thereby interact with one another.

In many examples, the electronic device may keep and display time, essentially functioning as a wristwatch among other things. Time may be displayed in an analog or digital format, depending on the device, its settings, and (in some cases) a user's preferences. Typically, time is displayed on a digital display stack forming part of the exterior of the device.

The display stack may include a cover element, such as a cover glass, overlying a display. The cover glass need not necessarily be formed from glass, although that is an option; it may be formed from sapphire, zirconia, alumina, chemically strengthened glass, hardened plastic and so on. Likewise, the display may be a liquid crystal display, an organic light-emitting diode display, or any other suitable display technology. Among other elements, the display stack may include a backlight in some examples.

The device may also include one or more touch sensors to determine a location of a touch on the cover glass. A touch sensor may be incorporated into or on the display stack in order to determine a location of a touch. The touch sensor may be self-capacitive in certain examples, mutual-capacitive in others, or a combination thereof.

Similarly, the device may include a force sensor to determine an amount of force applied to the cover glass. The force sensor may be a capacitive sensor in some examples and a strain sensor in other examples. In either example, the force sensor is generally transparent and made from transparent materials, or is located beneath or away from the display in order not to interfere with the view of the display. The force sensor may, for example, take the form of two capacitive plates separated by silicone or another deformable material. As the capacitive plates move closer together under an external force, the change in capacitance may be measured and a value of the external force correlated from the capacitance change. Further, by comparing relative capacitance changes from multiple points on the force sensor, or from multiple force sensors, a location or locations at which force is exerted may be determined. In one example the force sensor may take the form of a gasket extending beneath the periphery of the display. The gasket may be segmented or unitary, depending on the example.

The electronic device may also provide alerts to a user. An alert may be generated in response to: a change in status of the device (one example of which is power running low); receipt of information by the device (such as receiving a message); communications between the device and another mechanism/device (such as a second type of device informing the device that a message is waiting or communication is in progress); an operational state of an application (such as, as part of a game, or when a calendar appointment is imminent) or the operating system (such as when the device powers on or shuts down); and so on. The number and types of triggers for an alert are various and far-ranging.

The alert may be auditory, visual, haptic, or a combination thereof. A haptic actuator may be housed within the device and may move linearly to generate haptic output (although in alternative examples the haptic actuator may be rotary or any other type). A speaker may provide auditory components of an alert and the aforementioned display may provide visual alert components. In some examples a dedicated light, display, or other visual output component may be used as part of an alert.

The auditory, haptic, and/or visual components of the alert may be synchronized to provide an overall experience to a user. One or more components may be delayed relative to other components to create a desired synchronization among them. The components may be synchronized so that they are perceived substantially simultaneously; as one example, a haptic output may be initiated slightly before an auditory output since the haptic output may take longer to be perceived than the audio. As another example, a haptic output (or portion thereof) may be initiated substantially before the auditory output, but at a weak or even subliminal level, thereby priming the wearer to receive the auditory output.

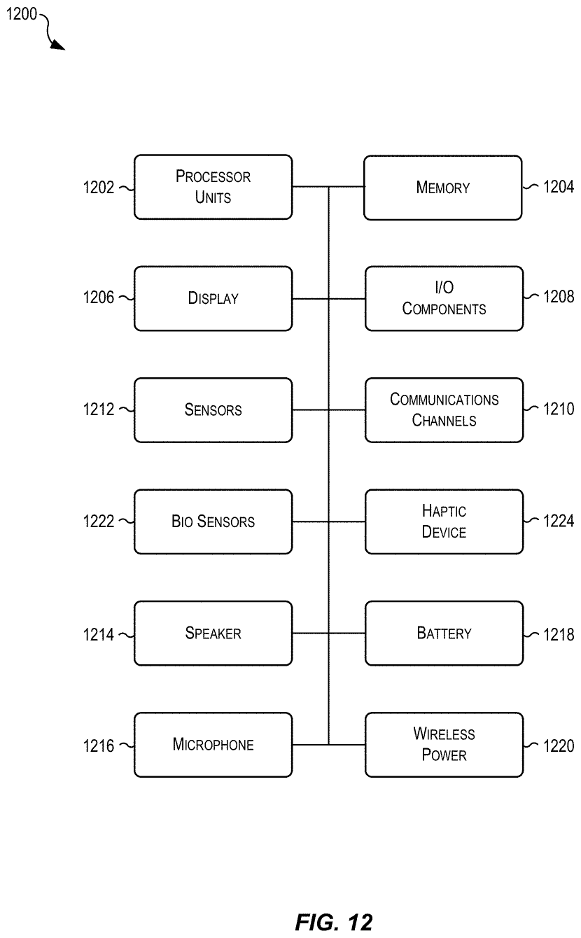

FIG. 12 depicts an example schematic diagram of a wearable electronic device 1200. The wearable electronic device 1200 is an example of the wearable user device 805. As shown in FIG. 12, the device 1200 includes one or more processing units 1202 that are configured to access a memory 1204 having instructions stored thereon.

Memories 1204, both removable and non-removable, are all examples of non-transitory computer-readable storage media. For example, non-transitory computer readable storage media may include volatile or non-volatile, removable or non-removable media implemented in any method or technology for storage of information such as computer-readable instructions, data structures, program modules, or other data. The memory 1204 is an example of non-transitory computer storage media. Additional types of computer storage media that may be present in the user device 104 may include, but are not limited to, phase-change RAM (PRAM), SRAM, DRAM, RAM, ROM, EEPROM, flash memory or other memory technology, compact disc read-only memory (CD-ROM), digital video disc (DVD) or other optical storage, magnetic cassettes, magnetic tape, magnetic disk storage or other magnetic storage devices, or any other medium that can be used to store the desired information and that can be accessed by the user device 1200. Combinations of any of the above should also be included within the scope of non-transitory computer-readable storage media. Alternatively, computer-readable communication media may include computer-readable instructions, program modules, or other data transmitted within a data signal, such as a carrier wave, or other transmission. However, as used herein, computer-readable storage media does not include computer-readable communication media.

The instructions or computer programs may be configured to perform one or more of the operations or functions described with respect to the device 1200 (e.g., the health application 1010(2)). For example, the instructions may be configured to control or coordinate the operation of the various components of the device. Such components include, but are not limited to, display 1206, one or more input/output components 1208, one or more communication channels 1210, one or more sensors 1212, a speaker 1214, microphone 1216, a battery 1218, wireless power 1220, bio sensors 1222, and/or one or more haptic feedback devices 1224. In some examples the speaker and microphone may be combined into a single unit and/or may share a common port through a housing of the device.

The processing units 1202 of FIG. 12 may be implemented as any electronic device capable of processing, receiving, or transmitting data or instructions. For example, the processing units 1202 may include one or more of: a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitably configured computing element or elements.

As shown in FIG. 12, the device 1200 may also include one or more acoustic elements, including a speaker 1214 and/or a microphone 1216. The speaker 1214 may include drive electronics or circuitry and may be configured to produce an audible sound or acoustic signal in response to a command or input. Similarly, the microphone 1216 may also include drive electronics or circuitry and is configured to receive an audible sound or acoustic signal in response to a command or input. The speaker 1214 and the microphone 1216 may be acoustically coupled to a port or opening in the case that allows acoustic energy to pass, but may prevent the ingress of liquid and other debris.

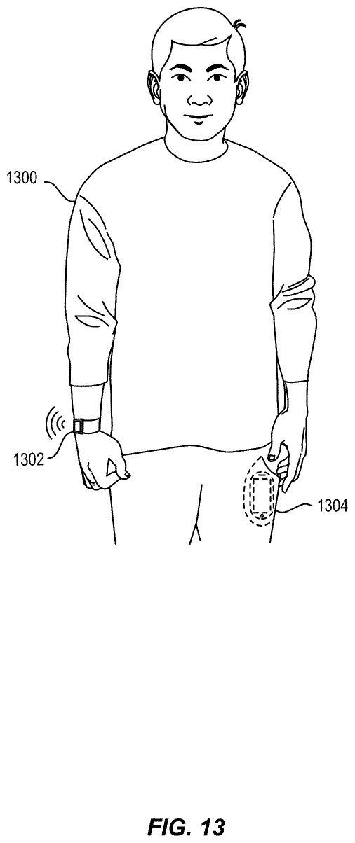

The example electronic device may communicate with other electronic devices either through a wired connection or wirelessly. Data may be passed between devices, permitting one device to relay information to another; control another; employ another's sensors, outputs, and/or inputs; and so on. FIG. 13 depicts a user 1300 wearing a first electronic device 1302 with a second electronic device 1304 in his pocket. Data may be wirelessly transmitted between the electronic devices 1302, 1304, thereby permitting the user 1300 to receive, view, and interact with data from the second device 1304 by means of the first electronic device 1302. Thus, the user 1300 may have access to part or all of the second device's functionality through the first electronic device 1302 without actually needing to interact directly with the second device 1304. In some examples, the second electronic device 1304 may be an example of the user device 802. The first electronic device 1302 may be an example of the wearable user device 805.

Further, the electronic devices 1302, 1304 may cooperate not only to share data, but to share functionality as well. For example, one of the two devices may incorporate a sensor, application, or function that the other lacks. The electronic device lacking such capabilities may request them from the other device, which may share wirelessly with the requesting device. Thus, multiple devices may operate together to provide expanded functions, software, access, and the like between the two and ultimately to a user. As one non-limiting example, the electronic device 1302 may be unable to place or receive telephone calls while the second device 1304 may be able to do so. A user may nonetheless make and/or receive calls through the first device 1302, which may employ the second device 1304 to actually place or accept a call.

As another non-limiting example, an electronic device 1302 may wirelessly communicate with a sales terminal nearby, thus permitting a user to quickly and efficiently conduct a transaction such as selling, buying, or returning a good. The electronic device may use near field communications technology to perform these and other functions.

As mentioned above, a band may be connected to two electronic devices and may serve as a wired communication path between the two. As another example, the devices may communicate wirelessly, thereby permitting one device to relay information from a second to a user. This latter example may be particularly useful when the second is inaccessible.

Certain examples may incorporate one or more biometric sensors to measure certain physiological characteristics of a user. The device may include a photoplesymogram sensor to determine a user's heart rate or blood oxygenation levels, for example. The device may also or instead include electrodes to measure the body impedance of a user, which may permit the device to estimate body fat percentages, the body's electrical activity, body impedance, and so on. Also include blood pressure, ultraviolet exposure, etc. Depending on the sensors incorporated into or associated with the electronic device, a variety of user characteristics may be measured and/or estimated, thereby permitting different health data to be provided to a user. In some examples, the sensed biometric data may be used, in part, to determine the historic, current, and/or predicted activity data of the user.

Certain examples may be wirelessly charged. For example, an inductive charging base may transmit power to an inductive receiver within the device in order to charge a battery of the device. Further, by varying the inductive field between the device and base, data may be communicated between the two. As one simple non-limiting example, this may be used to wake the base from a low-power sleep state to an active charging state when the device is placed on the base. Other wireless charging systems may also be used (e.g., near field magnetic resonance and radio frequency). Alternatively, the device may also employ wired charging through electrodes.

In certain examples, the device may include a rotary input, which may take the form of a crown with a stem. The crown and stem may be rotated to provide the rotary input. Rotation of the stem and/or crown may be sensed optically, electrically, magnetically, or mechanically. Further, in some examples the crown and stem may also move laterally, thereby providing a second type of input to the device.

The electronic device may likewise include one or more buttons. The button(s) may be depressed to provide yet another input to the device. In various examples, the button may be a dome switch, rocker switch, electrical contact, magnetic switch, and so on. In some examples the button may be waterproof or otherwise sealed against the environment.

Various examples may include or otherwise incorporate one or more motion sensors. A motion sensor may detect motion of the device and provide, modify, cease, or otherwise affect a state, output, or input of the device or associated applications based at least in part on the motion. As non-limiting examples, a motion may be used to silence the device or acknowledge an alert generated by the device. Sample motion sensors include accelerometers, gyroscopic sensors, magnetometers, GPS sensors, distance sensors, and so on. Some examples may use a GPS sensor to facilitate or enable location and/or navigation assistance.

Certain examples may incorporate an ambient light sensor. The ambient light sensor may permit the device to sense a brightness of its environment and adjust certain operational parameters accordingly. For example, the electronic device may modify a brightness of a display in response to the sensed ambient light. As another example, the electronic device may turn the display off if little or no light is sensed for a period of time.

These and other functions, operations, and abilities of the electronic device will be apparent upon reading the specification in its entirety.

Certain examples of a wearable electronic device may include one or more sensors that can be used to calculate a health metric or other health-related information. As one example, a wearable electronic device may function as a wearable health assistant that provides health-related information (whether real-time or not) to the user, authorized third parties, and/or an associated monitoring device.

FIG. 12 depicts an example electronic device 1200 having one or more biometric sensors. The electronic device 1200 is an example of the wearable user device 805. As shown in FIG. 12, an array of light sources and a photodetector 1251-1254 may be disposed on the rear surface of the device 1200. In one example, the light sources 1251-1253 are formed from light emitting diode (LED) elements that are configured to emit light into a portion of the wearer's body (e.g., wrist). The photodetector 1254 is shared between the multiple light sources 1251-1253 and is configured to receive light reflected from the body. The photodetector may be formed from a photodiode material that is configured to produce a signal based at least in part on the received light. In one implementation, the signal produced by the photodetector 1254 is used to compute a health metric associated with the wearer. In some cases, the light sources 1251-1253 and the photodetector 1254 form a photoplethysmography (PPG) sensor. The first light source 1251 may include, for example, a green LED, which may be adapted for detecting blood perfusion in the body of the wearer. The second light source 1252 may include, for example, an infrared LED, which may be adapted to detect changes in water content or other properties of the body. The third 1253 light source may be a similar type or different types of LED element, depending on the sensing configuration. The optical (e.g., PPG) sensor or sensors may be used to compute various health metrics, including, without limitation, a heart rate, a respiration rate, blood oxygenation level, a blood volume estimate, blood pressure, or a combination thereof. One or more of the light sources 1251-1253 and the photodetector 1254 may also be used for optical data transfer with a base or other device. While FIG. 12 depicts one example, the number of light sources and/or photodetectors may vary in different examples. For example, another example may use more than one photodetector. Another example may also use fewer or more light sources than are depicted in the example of FIG. 12.

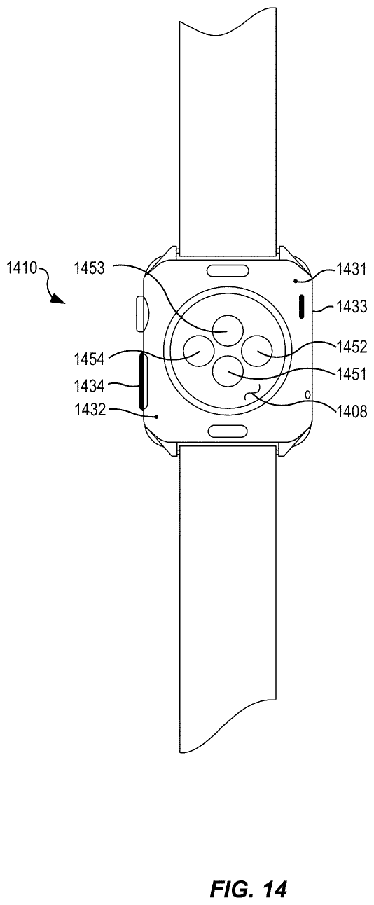

Also as shown in FIG. 14, the device 1400 includes multiple electrodes 1431, 1432, 1433, 1434 that are located on or near external surfaces of the device 1400. In the present example, the device 1400 includes a first electrode 1431 and a second electrode 1432 that are located on or proximate to a rear-facing surface of the device body 1410. In this example, the first electrode 1431 and the second electrode 1432 are configured to make electrical contact with the skin of the user wearing the device 1400. In some cases, the first 1431 and second 1432 electrodes are used to take an electrical measurement or receive an electrical signal from the body of the user. As also shown in FIG. 14, the device 1400 may include a third electrode 1433 and a fourth electrode 1434 that are located on or proximate to a perimeter of the case of the device body 1410. In the present example, the third 1433 and fourth 1434 electrodes are configured to be contacted by one or more fingers of the user who is wearing or interacting with the device 1400. In some cases, the third 1433 and fourth 1434 electrodes are also used to take an electrical measurement or receive an electrical signal from the body of the user. In some cases, the first 1431, second 1432, third 1433, and fourth 1434 electrodes are all used to take a measurement or series of measurements that can be used to compute another health metric of the user's body. Health metrics that may be computed using the electrodes include, without limitation, heart functions (ECG, EKG), water content, body-fat ratios, galvanic skin resistance, and combinations thereof.

In the configuration depicted in FIG. 14, the electronic device 1400 includes one or more apertures in the case 1410. A light source 1451-1454 may be disposed in each aperture. In one example, each light source 1451-1453 is implemented as a light-emitting diode (LED). In the present example, the four apertures, three light sources 1451-1453, and a single detector 1454 are used to form one or more sensors. Other examples can include any number of light sources. For example, two light sources can be used in some examples.

The light sources may operate at the same light wavelength range, or the light sources can operate at different light wavelength ranges. As one example, with two light sources, one light source may transmit light in the visible wavelength range while the other light source can emit light in the infrared wavelength range. With four light sources, two light sources may transmit light in the visible wavelength range while the other two light sources can emit light in the infrared wavelength range. For example, in one example, at least one light source can emit light in the wavelength range associated with the color green while another light source transmits light in the infrared wavelength range. When a physiological parameter of the user is to be determined, the light sources emit light toward the user's skin and the optical sensor senses an amount of reflected light. In some cases, a modulation pattern or sequence may be used to turn the light sources on and off and sample or sense the reflected light.

In the following, further clauses are described to facilitate the understanding of the present disclosure.

Clause 1. A computer-implemented method, comprising:
responsive to a first user input at a first user device, identifying a second user associated with a second user device to receive health data updates associated with a first user of the first user device;
responsive to a second user input at the first user device, identifying a set of authorization identifiers that each represent a type of health data update to be shared with the second user device;
generating a sharing request that identifies the set of authorization identifiers and a unique identifier that identifies the second user; and
sharing information associated with the sharing request with the second user device.

Clause 2. The computer-implemented method of clause 1, wherein individual authorization identifiers of the set of authorization identifiers correspond to health topics.

Clause 3. The computer-implemented method of clause 1, further comprising receiving a message indicating acceptance of the sharing request by the second user.

Clause 4. The computer-implemented method of clause 1, wherein sharing the information associated with the sharing request with the second user device comprises sending the information to a server, the information configured to enable the server to send the sharing request to the second user device.

Clause 5. The computer-implemented method of clause 1, further comprising:
generating a set of health data updates based at least in part on the set of authorization identifiers; and
combining the set of health data updates into a transaction.

Clause 6. The computer-implemented method of clause 5, further comprising, prior to generating the set of health data updates, detecting occurrence of a triggering event.

Clause 7. The computer-implemented method of clause 6, wherein the triggering event comprises at least one of opening of a health application on the first user device, collection of a type of health data associated with the set of authorization identifiers, collection of a certain quantity of health data associated with the set of authorization identifiers, achievement of a health-based goal associated with the set of authorization identifiers, or a trend in health data associated with the set of authorization identifiers.

Clause 8. The computer-implemented method of clause 5, further comprising sending information associated with the transaction to a server to enable the server to store the transaction in accordance with a storage zone associated with the unique identifier that identifies the second user.

Clause 9. The computer-implemented method of clause 5, wherein each health data update of the set of health data updates comprises source data and update metadata, and wherein the transaction comprises the set of health data updates and transaction metadata.

Clause 10. The computer-implemented method of clause 9, wherein the update metadata is useable by the second user device to determine display of the set of health data updates, and the transaction metadata is useable by the second user device to pick between different transactions.

Clause 11. The computer-implemented method of clause 1, wherein the unique identifier comprises a sharing identifier useable for uploading and downloading data from a server.

Clause 12. The computer-implemented method of clause 1, further comprising:
generating a different sharing request that identifies a different set of authorization identifiers and a different unique identifier that identifies a third user of a third user device, and
sending information associated with the different sharing request to a server, the information configured to enable the server to send the different sharing request to the third user device.

Clause 13. The computer-implemented method of clause 12, wherein the different set of authorization identifiers is distinct from the set of authorization identifiers.

Clause 14. The computer-implemented method of clause 1, wherein the health data updates are based on health data collected by or generated by at least one of the first user device or an accessory device of the first user device.

Clause 15. The computer-implemented method of clause 14, wherein the first user device comprises a handheld user device and the accessory device comprises a wearable user device.

Clause 16. One or more non-transitory computer-readable storage devices comprising computer-executable instructions that, when executed by a computer system, cause the computer system to perform the method of clauses 1-15.

Clause 17. A system, comprising:
one or more memories for storing computer-executable instructions; and
one or more processors configured to access the one or more memories and execute the computer-executable instructions to perform the method of any of clauses 1-15.

Clause 18. A computer-implemented method, comprising:
receiving, at a first user device associated with a first user, a sharing request associated with sharing health data of a second user associated with a second user device with the first user device;
requesting, from a server, transaction data stored by a server;
in response to requesting the transaction data, receiving, from the server, a first transaction package comprising first one or more health data updates associated with the health data of the second user;
extracting the first one or more health data updates from the first transaction package; and
populating a user interface of the first user device with information associated with the first one or more health data updates.

Clause 19. The computer-implemented method of clause 18, wherein the sharing request comprises a unique identifier of the first user.

Clause 20. The computer-implemented method of clause 18, further comprising, in response to requesting the transaction data, receiving, from the server, a second transaction package comprising second one or more health data updates associated with the health data of the second user associated with a third user device.

Clause 21. The computer-implemented method of clause 20, further comprising:
selecting one of the first transaction package or the second transaction package as a selected transaction package based at least in part on a set of transaction selection rules, and wherein:
extracting the first one or more health data updates from the first transaction package comprises extracting selected one or more health data updates from the selected transaction package; and
populating the user interface of the first user device with information associated with the first one or more health data updates comprises populating the user interface of the first user device with information associated with the selected one or more health data updates.

Clause 22. The computer-implemented method of clause 21, wherein selecting one of the first transaction package or the second transaction package as the selected transaction package based at least in part on the set of transaction selection rules comprises performing at least one of:
determining collection time information for each of the first and second transaction packages based at least in part on transaction metadata associated respectively with each of the first and second transaction packages;
determining application version information for each of the first and second transaction packages based at least in part on the transaction metadata respectively associated with each of the first and second transaction packages;
determining accessory device information for each of the first and second transaction packages based at least in part on the transaction metadata respectively associated with each of the first and second transaction packages; or
determining device identifier information associated with each of the second and third user devices used to collect the first and second transaction packages based at least in part on the transaction metadata respectively associated with each of the first and second transaction packages.

Clause 23. One or more non-transitory computer-readable storage devices comprising computer-executable instructions that, when executed by a computer system, cause the computer system to perform the method of clauses 18-22.

Clause 24. A system, comprising:
one or more memories for storing computer-executable instructions; and
one or more processors configured to access the one or more memories and execute the computer-executable instructions to perform the method of any of clauses 18-22.

Clause 25. A computer-implemented method, comprising:
determining a first change point in a first type of health data;
determining a second change point in a second type of health data that is correlated with the first type of health data;
in accordance with a determination that the first change point meets a first set of notification criteria, generating a notification that includes first information associated with the first change point; and
in accordance with a determination that the second change point meets a second set of notification criteria, including second information associated with the second change point in the notification.

Clause 26. The computer-implemented method of clause 25, wherein determining the first change point comprises comparing first health data of the first type of health data from a first time period with second health data of the first type of health data from a second time period that overlaps with the first time period.

Clause 27. The computer-implemented method of clause 25, wherein the first change point comprises a change in the first type of health data over a given time period.

Clause 28. The computer-implemented method of clause 25, further comprising, after determining the first change point and before determining the second change point, picking the second type of health data to evaluate based at least in part on a predefined correlation between the first and second types of health data.

Clause 29. The computer-implemented method of clause 25, wherein the first set of notification criteria is independent of the second type of health data and the second set of notification criteria is dependent on the first type of health data.

Clause 30. The computer-implemented method of clause 25, wherein:
determining the first change point in the first type of health data comprises determining the first change point in accordance with a first threshold; and
determining the second change point in the second type of health data comprises determining the second change point in accordance with a second threshold.

Clause 31. The computer-implemented method of clause 30, wherein the first threshold is an absolute threshold and the second threshold is a relative threshold.

Clause 32. The computer-implemented method of clause 31, wherein the relative threshold depends at least in part on the first change point occurring prior to the second change point.

Clause 33. The computer-implemented method of clause 25, further comprising providing the notification for presentation at a user device.

Clause 34. The computer-implemented method of clause 25, wherein first health data of the first type of health data is collected by at least one of a handheld user device or a wearable user device.

Clause 35. The computer-implemented method of clause 25, wherein the first set of notification criteria comprise a recency criterion defining a time threshold between sending notifications associated with the first type of health data, and a trend criterion defining conditions under which a change in direction of a trend in the first type of health data will trigger a notification.

Clause 36. The computer-implemented method of clause 25, further comprising, in accordance with a determination that second change point does not meet the second set of notification criteria, refraining from including the second information associated with the second change point from the notification.

Clause 37. The computer-implemented method of clause 25, wherein the first type of health data and the second type of health data are associated with a single user profile and are indicative of health of a user associated with the single user profile.

Clause 38. The computer-implemented method of clause 25, wherein:
determining the first change point in the first type of health data comprises determining the first change point at a first time; and
determining the second change point in the second type of health data comprises determining the second change point at a second time following the first time.

Clause 39. The computer-implemented method of clause 25, wherein:
determining that the first change point meets the first set of notification criteria comprises determining, at a first time, that the first change point meets the first set of notification criteria, and
determining that the second change point meets the second set of notification criteria comprises determining that the second change point is likely to occur within a threshold amount of time following the first time.

Clause 40. One or more non-transitory computer-readable storage devices comprising computer-executable instructions that, when executed by a computer system, cause the computer system to perform the method of clauses 25-39.

Clause 41. A system, comprising:
one or more memories for storing computer-executable instructions; and
one or more processors configured to access the one or more memories and execute the computer-executable instructions to perform the method of any of clauses 25-39.

Clause 42. A computer-implemented method, comprising:
accessing first health data of a first data type collected during a time window;
comparing a first quantity of the first health data associated with a first time sub-window of the time window with a second quantity of the first health data associated with a second time sub-window of the time window to identify a first change in the first health data;
in accordance with a first evaluation of the first and second quantities of the first health data with respect to a first set of change point criteria, determining that the first change is a first change point in the first health data;
generating a notification that includes information about the first change point; and
including information, in the notification, about a second change point in second health data when a second evaluation of the second health data with respect to a second set of change point criteria is indicative of the second change point.

Clause 43. The computer-implemented method of clause 42, wherein the second health data is of a second data type that is distinct from the first data type and is collected during the time window.

Clause 44. The computer-implemented method of clause 43, wherein the first set of change point criteria comprises a first set of thresholds and the second set of change point criteria comprises a second set of thresholds that is distinct from the first set of thresholds.

Clause 45. The computer-implemented method of clause 44, wherein the second set of thresholds is relaxed with respect to the first set of thresholds.

Clause 46. The computer-implemented method of clause 42, wherein the first set of change point criteria comprises a first statistical significance criterion and a first statistical magnitude criterion, and the second set of change point criteria comprises a second statistical significance criterion and a second statistical magnitude criterion.

Clause 47. The computer-implemented method of clause 42, wherein the time window comprises at least one of about one month or about six months.

Clause 48. The computer-implemented method of clause 42, further comprising iteratively evaluating portions of the first health data from differently-sized sub-windows within the time window to define the first time sub-window and the second time sub-window.

Clause 49. The computer-implemented method of clause 42, wherein the first change point comprises a data point in the first time window when the first health data is indicative of an upward trend or a downward trend.

Clause 50. The computer-implemented method of clause 42, wherein the first change point is statistically correlated with the second change point.

Clause 51. The computer-implemented method of clause 42, further comprising providing the notification for presentation at a display of a user device.

Clause 52. The computer-implemented method of clause 51, further comprising refraining from generating additional notifications for a fixed period time following providing the notification for presentation at the display of the user device.

Clause 53. The computer-implemented method of clause 42, wherein the notification comprises one or more graphical elements corresponding to at least one of the first change point or the second change point.

Clause 54. The computer-implemented method of clause 42, wherein the first health data and the second health data are collected by one or more sensors of a user device, and wherein the computer-implemented is performed by the user device.

Clause 55. The computer-implemented method of clause 42, wherein a first change point criterion of the first set of change point criteria is unique to the first data type and a second change point criterion of the first set of change point criteria is common with a corresponding third change point criterion of the second set of change point criteria.

Clause 56. The computer-implemented method of clause 42, further comprising:
comparing a third quantity of the first health data associated with a third time sub-window of the time window with a fourth quantity of the first health data associated with a fourth time sub-window of the time window to identify a third change in the first health data;
in accordance with a third evaluation of the third and fourth quantities of the first health data with respect to the first set of change point criteria, determining that the third change is a third change point in the first health data; and
selecting between the first change point and the third change point based on respective magnitudes of the first change and the third change, and wherein generating the notification comprises generating the notification that includes information about the selected change point.

Clause 57. The computer-implemented method of clause 42, wherein the first and second health data are associated with a user, and wherein the first change point and the second change point are related to a change in behavior of the user relating to health.

Clause 58. The computer-implemented method of clause 42, wherein the first health data are collected by a first user device associated with a first user, and wherein accessing the first health data comprises receiving, by a second user device associated with a second user, the first health data from the first user device.

Clause 59. The computer-implemented method of clause 58, further comprising providing the notification for presentation at the second user device.

Clause 60. The computer-implemented method of clause 58, wherein receiving the first health data from the first user device comprises the second user device requesting the first health data from a cloud-based server that stores the first health data on behalf of the first user.

Clause 61. One or more non-transitory computer-readable storage devices comprising computer-executable instructions that, when executed by a computer system, cause the computer system to perform the method of clauses 42-60.

Clause 62. A system, comprising:
one or more memories for storing computer-executable instructions; and
one or more processors configured to access the one or more memories and execute the computer-executable instructions to perform the method of any of clauses 42-60.

Illustrative methods and systems for managing user device connections are described above. Some or all of these systems and methods may, but need not, be implemented at least partially by architectures such as those shown at least in FIGS. 1-14. While many of the examples are described above with reference to personal, activity, and/or health-related information, it should be understood that any type of user information or non-user information (e.g., data of any type) may be managed using these techniques. Further, in the foregoing description, various non-limiting examples were described. For purposes of explanation, specific configurations and details are set forth in order to provide a thorough understanding of the examples. However, it should also be apparent to one skilled in the art that the examples may be practiced without the specific details. Furthermore, well-known features were sometimes omitted or simplified in order not to obscure the example being described.

The various examples further can be implemented in a wide variety of operating environments, which in some cases can include one or more user computers, computing devices or processing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system also can include a number of workstations running any of a variety of commercially available operating systems and other known applications for purposes such as development and database management. These devices also can include other electronic devices, such as dummy terminals, thin-clients, gaming systems, and other devices capable of communicating via a network.

Most examples utilize at least one network that would be familiar to those skilled in the art for supporting communications using any of a variety of commercially available protocols, such as TCP/IP, OSI, FTP, UPnP, NFS, CIFS, and AppleTalk. The network can be, for example, a local area network, a wide-area network, a virtual private network, the Internet, an intranet, an extranet, a public switched telephone network, an infrared network, a wireless network, and any combination thereof.

In examples utilizing a network server, the network server can run any of a variety of server or mid-tier applications, including HTTP servers, FTP servers, CGI servers, data servers, Java servers, and business application servers. The server(s) may also be capable of executing programs or scripts in response to requests from user devices, such as by executing one or more applications that may be implemented as one or more scripts or programs written in any programming language, such as Java®, C, C# or C++, or any scripting language, such as Perl, Python or TCL, as well as combinations thereof. The server(s) may also include database servers, including without limitation those commercially available from Oracle °, Microsoft®, Sybase®, and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of examples, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen, or keypad), and at least one output device (e.g., a display device, printer, or speaker). Such a system may also include one or more storage devices, such as disk drives, optical storage devices, and solid-state storage devices such as RAM or ROM, as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a non-transitory computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or browser. It should be appreciated that alternate examples may have numerous variations from that described above. For example, customized hardware might also be used and/or particular elements might be implemented in hardware, software (including portable software, such as applets) or both. Further, connection to other computing devices such as network input/output devices may be employed.

Non-transitory storage media and computer-readable media for containing code, or portions of code, can include any appropriate media known or used in the art, including storage media, such as, but not limited to, volatile and non-volatile, removable and non-removable media implemented in any method or technology for storage of information such as computer-readable instructions, data structures, program modules, or other data, including RAM, ROM, EEPROM, flash memory or other memory technology, CD-ROM, DVD or other optical storage, magnetic cassettes, magnetic tape, magnetic disk storage or other magnetic storage devices, or any other medium which can be used to store the desired information and which can be accessed by a system device. Based at least in part on the disclosure and teachings provided herein, a person of ordinary skill in the art will appreciate other ways and/or methods to implement the various examples.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated examples thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a” and “an” and “the” and similar referents in the context of describing the disclosed examples (especially in the context of the following claims) are to be construed to cover both the singular and the plural, unless otherwise indicated herein or clearly contradicted by context. The terms “comprising,” “having,” “including,” and “containing” are to be construed as open-ended terms (e.g., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate examples of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain examples require at least one of X, at least one of Y, or at least one of Z to each be present.

Preferred examples of this disclosure are described herein, including the best mode known to the inventors for carrying out the disclosure. Variations of those preferred examples may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

As described above, one aspect of the present technology is sharing health data updates between user devices, which may include storing some aspect of the data on a server. The present disclosure contemplates that in some instances, this gathered data may include personally identifiable information (PII) data that uniquely identifies or can be used to contact or locate a specific person. Such personal information data can include demographic data, location-based data, telephone numbers, email addresses, Twitter IDs, home addresses, data or records relating to a user's health or level of fitness (e.g., vital sign measurements, medication information, exercise information), date of birth, health record data, or any other identifying or personal or health information.

The present disclosure recognizes that the use of such personal information data, in the present technology, can be used to the benefit of users. For example, the personal information data can be used to provide a family member or friend a view of health data updates. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the U.S., collection of or access to certain health data may be governed by federal and/or state laws, such as the Health Insurance Portability and Accountability Act (HIPAA); whereas health data in other countries may be subject to other regulations and policies and should be handled accordingly. Hence, different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services or other services relating to health record management, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health-related applications, data de-identification can be used to protect a user's privacy. De-identification may be facilitated, when appropriate, by removing specific identifiers (e.g., date of birth), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

What is claimed is:

1. A computer-implemented method, comprising:
accessing first health data of a first data type collected during a time window;
comparing a first quantity of the first health data associated with a first time sub-window of the time window with a second quantity of the first health data associated with a second time sub-window of the time window to identify a first change in the first health data;
in accordance with a first evaluation of the first and second quantities of the first health data with respect to a first set of change point criteria, determining that the first change is a first change point in the first health data;
generating a notification that includes information about the first change point; and
including information, in the notification, about a second change point in second health data when a second evaluation of the second health data with respect to a second set of change point criteria is indicative of the second change point.

2. The computer-implemented method of claim 1, wherein the second health data is of a second data type that is distinct from the first data type and is collected during the time window.

3. The computer-implemented method of claim 2, wherein the first set of change point criteria comprises a first set of thresholds and the second set of change point criteria comprises a second set of thresholds that is distinct from the first set of thresholds.

4. The computer-implemented method of claim 3, wherein the second set of thresholds is relaxed with respect to the first set of thresholds.

5. The computer-implemented method of claim 1, wherein the first set of change point criteria comprises a first statistical significance criterion and a first statistical magnitude criterion, and the second set of change point criteria comprises a second statistical significance criterion and a second statistical magnitude criterion.

6. The computer-implemented method of claim 1, wherein the time window comprises at least one of about one month or about six months.

7. The computer-implemented method of claim 1, further comprising iteratively evaluating portions of the first health data from differently-sized sub-windows within the time window to define the first time sub-window and the second time sub-window.

8. The computer-implemented method of claim 1, wherein the first change point comprises a data point in the first time sub window when the first health data is indicative of an upward trend or a downward trend.

9. One or more non-transitory computer-readable storage devices comprising computer-executable instructions that, when executed by one or more processors of a computer system, cause the computer system to perform operations comprising:
accessing first health data of a first data type collected during a time window;
comparing a first quantity of the first health data associated with a first time sub-window of the time window with a second quantity of the first health data associated with a second time sub-window of the time window to identify a first change in the first health data;
in accordance with a first evaluation of the first and second quantities of the first health data with respect to a first set of change point criteria, determining that the first change is a first change point in the first health data;
generating a notification that includes information about the first change point; and
including information, in the notification, about a second change point in second health data when a second evaluation of the second health data with respect to a second set of change point criteria is indicative of the second change point.

10. The one or more non-transitory computer-readable storage devices of claim 9, wherein the first change point is statistically correlated with the second change point.

11. The one or more non-transitory computer-readable storage devices of claim 9, further comprising additional computer-executable instructions that, when executed by the one or more processors, cause the computer system to perform additional operations comprising providing the notification for presentation at a display of a user device.

12. The one or more non-transitory computer-readable storage devices of claim 11, further comprising additional computer-executable instructions that, when executed by the one or more processors, cause the computer system to perform additional operations comprising refraining from generating additional notifications for a fixed period time following providing the notification for presentation at the display of the user device.

13. The one or more non-transitory computer-readable storage devices of claim 9, wherein the notification comprises one or more graphical elements corresponding to at least one of the first change point or the second change point.

14. The one or more non-transitory computer-readable storage devices of claim 9, wherein the first health data and the second health data are collected by one or more sensors of a user device.

15. The one or more non-transitory computer-readable storage devices of claim 9, wherein a first change point criterion of the first set of change point criteria is unique to the first data type and a second change point criterion of the first set of change point criteria is common with a corresponding third change point criterion of the second set of change point criteria.

16. The one or more non-transitory computer-readable storage devices of claim 9, further comprising additional computer-executable instructions that, when executed by the one or more processors, cause the computer system to perform additional operations comprising:
comparing a third quantity of the first health data associated with a third time sub-window of the time window with a fourth quantity of the first health data associated with a fourth time sub-window of the time window to identify a third change in the first health data;
in accordance with a third evaluation of the third and fourth quantities of the first health data with respect to the first set of change point criteria, determining that the third change is a third change point in the first health data; and
selecting between the first change point and the third change point based on respective magnitudes of the first change and the third change, and wherein generating the notification comprises generating the notification that includes information about the selected change point.

17. The one or more non-transitory computer-readable storage devices of claim 9, wherein the first and second health data are associated with a user, and wherein the first change point and the second change point are related to a change in behavior of the user relating to health.

18. The one or more non-transitory computer-readable storage devices of claim 9, wherein the first health data are collected by a first user device associated with a first user, and wherein accessing the first health data comprises receiving, by a second user device associated with a second user, the first health data from the first user device.

19. The one or more non-transitory computer-readable storage devices of claim 18, wherein receiving the first health data from the first user device comprises the second user device requesting the first health data from a cloud-based server that stores the first health data on behalf of the first user.

20. A computer system, comprising:
a memory configured to store computer-executable instructions; and
a processor configured to access the memory and execute the computer-executable instructions to at least:
access first health data of a first data type collected during a time window;
compare a first quantity of the first health data associated with a first time sub-window of the time window with a second quantity of the first health data associated with a second time sub-window of the time window to identify a first change in the first health data;
in accordance with a first evaluation of the first and second quantities of the first health data with respect to a first set of change point criteria, determine that the first change is a first change point in the first health data;
generate a notification that includes information about the first change point; and
include information, in the notification, about a second change point in second health data when a second evaluation of the second health data with respect to a second set of change point criteria is indicative of the second change point.
